(19) United States
(12) Patent Application Publication
(10) Pub. No.: US 2016/0166701 A1
(43) Pub. Date: Jun. 16, 2016

(54) DELIVERY COMPOSITION FOR TOPICAL APPLICATIONS AND INJECTIONS AND OPHTHALMIC FORMULATIONS, METHODS FOR MANUFACTURING THEREOF, AND METHODS FOR DELIVERY OF A DRUG-DELIVERY COMPOSITION

(71) Applicant: THERAKINE BIODELIVERY GMBH, Berlin (DE)

(72) Inventors: Andreas VOIGT, Berlin (DE); Scott HAMPTON, Tarpon Springs, FL (US); Andreas REIFF, San Marino, CA (US); Mariana DOBRANIS, Berlin (DE)

(21) Appl. No.: 14/902,651

(22) PCT Filed: Jul. 4, 2014
(86) PCT No.: PCT/EP2014/064330

§ 371 Date: Jan. 4, 2016

Related U.S. Application Data
(60) Provisional application No. 61/843,117, filed on Jul. 5, 2013.

(51) Int. Cl. A61K 47/36; A61K 38/20; A61K 39/395

(52) U.S. Cl. CPC A61K 47/36; A61K 39/395; A61K 38/2006; A61K 39/3955

(57) ABSTRACT

A drug-delivery composition includes an intermediate composition having a hydrophilic matrix of a cross-linked polymer in form of particles, and a pharmaceutically active composition distributed in the cross-linked polymer of the particles.

### FIELD OF THE INVENTION

The present invention relates to the field of sustained drug release, particularly to drug release compositions comprising proteins as pharmaceutically active compounds for use in ophthalmic formulations, topical applications, and injections, methods for manufacturing said drug-delivery compositions, and to the sustained delivery thereof into living organisms and tissues for therapeutic purposes.

### BACKGROUND OF THE INVENTION

Over 40,000 corneal transplants are performed each year in the US due to chronic diseases such as infections, diabetes or facial injuries. Globally, corneal blindness affects more than 10 million people and thus far, the only treatment available is corneal transplantation.

However, the limited supply of donor tissue creates a significant need for alternative artificial corneas. A viable tissue-engineered (TE) corneal equivalent created partly from patient-derived corneal cells must be transparent, but to date despite much effort artificial corneas remains elusive. Several other attempts have been made to develop compositions which enable to solve at least part of the above-mentioned problems, but so far with little success.

Particularly, current prophylactic and therapeutic regimens for corneal injuries, whether traumatic or surgically induced, are often associated with significant complications such as uncoordinated healing and scar formation, infections, post-surgical overcorrection, under correction, halo formation and scatter effects.

In addition, eye injuries remain common combat injuries due to roadside bombs, mortars and grenades. Protective gear, only offers limited protection. Often, the face is unprotected, leading to serious facial trauma (including damage to the eyes and potential loss of sight).

Some of these soldiers will require corneal transplantation or laser in situ keratomileusis (LASIK), a type of refractive surgery for the correction of myopia, hypermetropia, and astigmatism.

De Brouwere D, Ginis H, Kymionis G, Naoumidi I, Pallikaris I.: Forward scattering properties of corneal haze. Optom Vis Sci. 2008 September; 85 (9): 843-8 refers to animal studies comparing various concentrations of topical IL-1ra against steroid and saline eye drop controls following photorefractive keratectomy (PRK) in rabbits. PRK was used to simulate corneal injury with 70 micron to 100-micron ablation of the central 6 mm optical zone. Each IL-1ra-based treatment group received topical antibiotics (moxifloxacin, 0.5%) to reduce the risk of infection, followed by a specific concentration of IL-1ra in a balanced salt solution (BSS). In all cases the treatment occurred four times daily and was continued until epithelial closure was complete. No serious adverse events such as infections or worsening infections in IL-1ra treated eyes were observed.

Davies B W, Panday V, Caldwell M, Scribbick F, Reilly C D: Effect of topical immunomodulatory interleukin 1 receptor antagonist therapy on corneal healing in New Zealand white rabbits (Oryctolagus cunniculus) after photorefractive keratectomy, Arch Ophthalmol. 2011 July; 129 (7): 909-13 describes a study concerning the effect of topical immunomodulatory interleukin 1 receptor antagonist therapy on corneal healing in New Zealand white rabbits (Oryctolagus cunniculus) after photorefractive keratectomy.

Thompson R E, Boraas L C, Sowder M, Bechtel M K, Orwin E J, Three-dimensional cell culture environment promotes partial recovery of the native corneal keratocyte phenotype from a subcultured population, Tissue Eng Part A. 2013 July; 19 (13-14): 1564-72 describes a study concerning the promotion of a partial recovery of the native corneal keratocyte phenotype from a subcultured population by a three-dimensional cell culture environment.

In view of the above, there is a need for simpler, safer, and more efficacious drug-delivery compositions that can promote corneal wound healing with significantly improved visual outcomes for patients affected by these injuries and untoward surgical results.

### SUMMARY OF THE INVENTION

According to an embodiment, a drug-delivery composition includes a liquid medium, and an intermediate composition, which includes flat shaped particles dispersed in the liquid medium. The particles include a hydrophilic matrix of covalently cross-linked glycosaminoglycans and a pharmaceutically active composition including a protein dispersed or distributed in the hydrophilic matrix of the particles, wherein the particles have an aspect ratio of from 2:1 to 50:1 and an average particle size of from 100 nm to 100 μm, and wherein the protein is an antibody or a therapeutic protein. The drug-delivery composition is for one of topical application, topical ophthalmic applications, and injectable application.

According to an embodiment, a drug-delivery composition includes an intermediate composition that includes a hydrophilic matrix, and a pharmaceutically active composition. The pharmaceutically active composition, which includes a protein, is dispersed or distributed in the hydrophilic matrix. The hydrophilic matrix is in the form of particles. The particles include cross-linked hyaluronic acid, have an aspect ratio of from 1:1 to 50:1, and have an average particle size of from 100 nm to 100 μm.

According to an embodiment, a drug-delivery composition includes an intermediate eye drop composition that includes a hydrophilic matrix, and a pharmaceutically active composition. The pharmaceutically active composition including a protein is dispersed or distributed in the hydrophilic matrix. Said hydrophilic matrix is in the form of particles, which include at least one cross-linked polymer, have an aspect ratio of from 1:1 to 50:1, and have an average particle size of from 500 nm to 5 μm.

According to an embodiment, a drug-delivery composition includes an intermediate composition, which includes a hydrophilic matrix, and a pharmaceutically active composition. The pharmaceutically active composition includes a protein, which is an antibody or a therapeutic protein. The pharmaceutically active composition including the antibody is dispersed or distributed in the hydrophilic matrix, which is provided in form of particles. The particles include at least one cross-linked polymer, have an aspect ratio of from 1:1 to 50:1, and have an average particle size of from 100 nm to 100 μm.

According to an embodiment, a drug-delivery composition includes an intermediate composition, which includes a hydrophilic matrix, and a pharmaceutically active composition.

The pharmaceutically active composition including a protein is dispersed or distributed in the hydrophilic matrix. The hydrophilic matrix is provided in form of particles which include at least one cross-linked polymer, have an aspect ratio of from 1:1 to 50:1, and have an average particle size of from 100 nm to 100 μm.

According to an embodiment, a method for manufacturing a drug-delivery composition is provided. The method includes providing a hydrophilic matrix and providing a pharmaceutically active composition. The pharmaceutically active composition includes a protein. The hydrophilic matrix is provided in form of particles. The particles include at least one cross-linked polymer. The method further includes mixing the hydrophilic matrix and the protein in a weight ratio from 1:1 to 50:1 to form an intermediate composition.

According to an embodiment, a method for manufacturing a drug-delivery composition includes providing a hydrophilic matrix, including at least one cross-linked polymer, in form of particles, providing a pharmaceutically active composition comprising a protein, mixing the hydrophilic matrix and the pharmaceutically active composition so that the protein is provided in a weight ratio from 1:1 to 1:20, particularly from 1:1 to 1:10, relative to the hydrophilic matrix to form an intermediate composition of the drug-delivery composition, wherein the intermediate composition comprises the particles and the protein distributed within the particles.

According to an embodiment, a method for manufacturing a drug-delivery composition includes providing a liquid film of a non-cross-linked polymer; drying the liquid film of the non-cross-linked polymer to obtain an at least partially dried film of the non-cross-linked polymer having a moisture content of less than 30%; adding a cross-linking agent to the at least partially dried film of the non-cross-linked polymer to cross-link the polymer; drying the film of the cross-linked polymer at an elevated temperature to obtain a dried film of the non-cross-linked polymer; and breaking the dried film of the cross-linked polymer to particles by a mechanical process to obtain particles having an aspect ratio of 1:1 to 50:1, particularly of 2:1 to 50:1, and an average particle size of 100 nm to 100 μm.

Those skilled in the art will recognize additional features and advantages upon reading the following detailed description, and upon viewing the accompanying drawings.

### BRIEF DESCRIPTION OF THE DRAWINGS

The accompanying drawings are included to give a better understanding of the embodiments that are incorporated in and constitute a part of this specification. The drawings illustrate embodiments and together with the description serve to explain principles of embodiments. Other embodiments and many of the intended advantages of embodiments will be readily appreciated, as they become better understood by reference to the following detailed description. The elements of the drawings are not necessarily to scale relative to each other.

FIG. 1 illustrates the different adsorption permanence comparing a model of collectives of cross-linked and of free molecules with focal contact sites. Every desorbed molecule has a large probability to leave the collective (right side). If the adsorbing molecules are chemically (covalently) cross-linked they can only leave the collective if all or at least a majority of them is becoming desorbed. The probability of this event is rather low.

FIG. 2 illustrates the release profile of antibodies from hyaluronic acid as hydrophilic matrix at room temperature.

FIG. 3 illustrates the release profile of antibodies from hyaluronic acid as hydrophilic matrix at room temperature.

FIG. 4 illustrates a picture of typical flat cross-linked hyaluronic acid particles.

FIG. 5 illustrates the results of corneal clarity and haze measurement in the various treatment groups according to Example 2.

FIG. 6 illustrates the mean Central Optical Density (microns) among the various treatment groups when compared to steroids using Pentacam exams over time divided in left (OS) and right eye (OD) according to Example 2.

FIG. 7 illustrates the thickness of the foam layer (in microns) as an undesirable outcome among the various treatment groups when compared to steroids according to Example 2.

FIG. 8 illustrates the redifferentiation of myofibroblasts to the quiescent keratocyte phenotype.

FIG. 9 illustrates preliminary confocal study results of cell grown in 3D collagen matrices with different amounts of the matrix formulation, according to Example 5.

FIG. 10 illustrates release profiles in different release media at room temperature for antibody material incorporated in a hyaluronic acid matrix according to the present invention.

### DETAILED DESCRIPTION OF THE INVENTION

Reference will now be made in detail to various aspects of the invention and embodiments. Each aspect is provided by way of explanation and is not meant as a limitation. For example, features illustrated or described as part of one aspect or embodiment can be used on or in conjunction with any other aspect or embodiment to yield yet a further aspect or embodiment. It is intended that the present disclosure includes any such combinations and variations.

In the following, if not otherwise defined, the term “% w/w” refers to the concentration by weight of a component (e.g. intermediate composition) based on the total weight of the respective entity (e.g. the total weight of drug-delivery composition). Furthermore, if not otherwise stated, all measurements were carried out at room temperature.

Moreover, unless otherwise defined, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired and intended properties. At the very least, and not as an attempt to limit the application of the doctrine of equivalents to the scope of the claims, each numerical parameter should at least be construed in light of the number of reported significant digits and by applying ordinary rounding techniques.

For the purpose of this application, the term “naturally occurring” intends to describe things existing in nature and exist without artificial aid. For instance, naturally occurring proteins are proteins which naturally exist in organisms, e.g. proteins which are encoded in humans without being modified in any contrivable way, e.g. by substituting one or more amino acids.

For the purpose of this application, if not otherwise stated, particle size is determined by microscopy. Osmolarity is determined by a Krüss Osmometer. The pH value is determined by using a pH meter (Mettler-Toledo). Moisture content is determined by using Karl Fischer titration (Metrohm 870 Titrino plus).

As a result of our intensive studies taking the above described problems into consideration, the present inventors were surprised to find that drug-delivery compositions comprising an intermediate composition comprising a specific hydrophilic matrix and a pharmaceutically active composition incorporated in said hydrophilic matrix provide a sustained drug release to be used in long-term treatment regimens of ophthalmic disorders.

According to an embodiment, a drug-delivery composition includes an intermediate composition. The intermediate composition includes a hydrophilic matrix in form of particles. The particles include at least one cross-linked polymer, have an aspect ratio of from 1:1 to 50:1, and have an average particle size of 100 nm to 100 μm. The intermediate composition further includes a pharmaceutically active composition that includes a protein which is dispersed in the hydrophilic matrix.

### Drug-Delivery Composition

For the purpose of this application, the term “drug-delivery composition” intends to describe any pharmaceutical dosage form known to those skilled in the art for transporting a pharmaceutically active compound into the human or animal body in order to achieve its desired therapeutic and/or diagnostic effects. Typically, pharmaceutical dosage forms comprise a mixture of a drug components, i.e. pharmaceutically active compound(s), and nondrug components (i.e. excipients). In general, these pharmaceutical dosage forms can be categorized by different aspects, e.g. their route of administration, (e.g. oral, inhalational, parenteral, topical administration) or their physical appearance (e.g. solid, semi-solid, liquid, gaseous). For the purpose of this application, particularly those semi-solid or liquid dosage forms are used, which can be administered topically or via parenteral injection. For instance, such dosage forms comprise ointments, creams, gels, lotions, dispersions, solutions or injection solutions, which is meant to be a non-exhaustive list of possible dosage forms.

According to an embodiment that can be combined with any of the other embodiments described herein, the drug-delivery composition comprises an intermediate composition. The concentration of the intermediate composition may be from 0.1% w/w to 70% w/w, particularly from 1% w/w to 70% w/w, particularly from 5% w/w to 60% w/w, particularly from 0.5% w/w to 40% w/w, more particularly from 1% w/w to 20% w/w, more particularly from 10% w/w to 40% w/w, based on the total weight of the drug-delivery composition.

Furthermore, the concentration of the intermediate composition can be from 10% w/w to 45% w/w, particularly from 20% w/w to 40% w/w, more particularly from 30% w/w to 38% w/w, based on the total weight of the drug-delivery composition. Also, the concentration of the intermediate composition can be from 1% w/w to 30% w/w, particularly from 5% w/w to 25% w/w, more particularly from 10% w/w to 20% w/w, based on the total weight of the drug-delivery composition. Also, the concentration of the intermediate composition may be from 0.1% w/w to 15% w/w, particularly from 1% w/w to 10% w/w, more particularly from 2% w/w to 5% w/w, based on the total weight of the drug-delivery composition.

According to an embodiment that can be combined with any of the other embodiments described herein, the drug-delivery composition further comprises a liquid medium.

According to an embodiment which can be combined with any of the other embodiments described herein the drug-delivery composition comprises the intermediate composition and the liquid medium in separated containers prior to administration to human or animal body.

According to an embodiment which can be combined with any of the other embodiments described herein, the liquid medium is selected from an aqueous solution, such as phosphate buffer saline (PBS), water, such as aqua ad injectabilia, or oils such as castor oil, clove oil, cassia oil, almond oil, corn oil, arachis oil, cottonseed oil, safflower oil, maize oil, linseed oil, rapeseed oil, soybean oil, caraway oil, rosemary oil, peanut oil, peppermint oil, sunflower oil, eucalyptus oil, olive oil, mentha oil, peppermint oil, eucalyptus oil, bergamot oil, anise oil, fennel oil, or rose oil. These liquid media can be used alone or in any combination of two or more kinds thereof. The concentration of the liquid medium may be from 30% w/w to 99.9% w/w, particularly from 30% w/w to 99% w/w, particularly from 40% w/w to 99% w/w, particularly from 40% w/w to 95% w/w, more particularly from 60% w/w to 90% w/w, based on the total weight of the drug-delivery composition.

Also, the concentration of the liquid medium can be from 55% w/w to 90% w/w, particularly from 60% w/w to 80% w/w, more particularly from 62% w/w to 70% w/w of liquid medium, based on the total weight of the drug-delivery composition. Also, the concentration of the liquid medium may be from 70% w/w to 99% w/w, particularly from 75% w/w to 95% w/w, more particularly from 80% w/w to 90% w/w, based on the total weight of the drug-delivery composition. Also, the concentration of the liquid medium can be from 85% w/w to 99.9% w/w, particularly from 90% w/w to 99% w/w, more particularly from 95% w/w to 98% w/w, based on the total weight of the drug-delivery composition.

According to an embodiment that can be combined with any of the other embodiments described herein, the drug-delivery composition further comprises at least one additive.

According to an embodiment which can be combined with any of the other embodiments described herein, and where at least one additive is selected from the group consisting of a soothing agent, a buffer agent, a preservative agent, a surfactant, a stabilizing agent, a tonicity agent and an antioxidant. These additives can be used alone or in any combination of two or more kinds thereof. For example, the drug-delivery composition can contain only one of the above additives or only two of these additives without any further additive of the above mentioned additives. For example, the drug-delivery composition can contain only a buffer agent without any further additive of the above mentioned additives. Furthermore, the drug-delivery composition can contain only a preservative agent without any further additive of the above mentioned additives. Moreover, the drug-delivery composition can contain only a surfactant and a buffer agent without any further additive of the above mentioned additives. Even further, the drug-delivery composition can contain only a tonicity agent and a buffer agent without any further additive of the above mentioned additives.

According to an alternative embodiment, the drug-delivery composition does not contain any of the above additives.

According to an embodiment, which can be combined with any of the other embodiments described herein, the soothing agent may include but is not limited to povidone, hydroxypropyl methyl cellulose, poloxamers, carboxymethyl cellulose, hydroxyethyl cellulose and glycerine. The concentration of soothing agent may be from 0.1% w/w to 2% w/w, particularly from 0.25% w/w to 0.7% w/w, more particularly from 0.3% w/w to 0.5% w/w, based on the total weight of the drug-delivery composition.

According to an embodiment that can be combined with any of the other embodiments described herein, the buffer agent may include but is not limited to, acetate buffers, citrate buffers, phosphate buffers, borate buffers, or a combination thereof. Acids or bases may be used to adjust the pH of these agents as needed. The concentration of buffer agent may be from 0.1% w/w to 2% w/w, particularly from 0.25% w/w to 0.7% w/w, more particularly from 0.3% w/w to 0.5% w/w, based on the total weight of the drug-delivery composition.

According to an embodiment that can be combined with any of the other embodiments described herein, the preservative agent may include any substance suitable for preventing microbial contamination in a drug-delivery composition subject to multiple uses from the same container. Suitable preservative agents may include, but are not limited to, cationic preservatives such as quaternary ammonium compounds including benzalkonium chloride, polyquad, and the like; guanidine-based preservatives including polyhexamethylene biguanide, chlorhexidine, and the like; chlorobutanol; mercury preservatives such as thimerosal, phenylmercuric acetate and phenylmercuric nitrate; and oxidizing preservatives such as stabilized oxychloro complexes. The concentration of preservative in the liquid may be from 0.0001% w/w to 25% w/w, particularly from 0.002% w/w to 0.05% w/w, more particularly from 0.005% w/w to 0.02% w/w, based on the total weight of the drug-delivery composition.

According to an embodiment that can be combined with any of the other embodiments described herein, the surfactant may include any compound that is surface active or can form micelles. A surfactant may be used for assisting in dissolving an excipient or an active agent, dispersing a solid or liquid in a composition, enhancing wetting, modifying drop size, stabilizing an emulsion, or a number of other purposes. Suitable surfactants may include, but are not limited to, alcohols; amine oxides; block polymers; carboxylated alcohol or alkylphenol ethoxylates; carboxylic acids/fatty acids; ethoxylated alcohols; ethoxylated alkylphenols; ethoxylated arylphenols; ethoxylated fatty acids; ethoxylated fatty esters or oils (animal and vegetable); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants; propoxylated and ethoxylated fatty acids, alcohols, or alkyl phenols; protein-based surfactants; sarcosine derivatives; sorbitan derivatives; sucrose and glucose esters and derivatives. The concentration of surfactant may be from 0.001% w/w to 5% w/w, particularly from 0.1% w/w to 2 w/w %, more particularly from 0.1% w/w to 1% w/w, based on the total weight of the drug-delivery composition.

According to an embodiment which can be combined with any of the other embodiments described herein, the stabilizing agent may include, but is not limited to, polyvinyl alcohol, povidone, hydroxypropyl methyl cellulose, poloxamers, carboxymethyl cellulose, hydroxyethyl cellulose, acrylates, mannitol, oligosaccharides, trehalose, protein-solution-stabilizing agents. In some embodiments, the concentration of stabilizing agent may be from 0.01% to 2%, particularly from 0.05% w/w to 1.5% w/w, more particularly from 0.1% w/w to 0.1, based on the total weight of the drug-delivery composition.

According to an embodiment that can be combined with any of the other embodiments described herein, the tonicity agent may include any compound or substance useful for adjusting the tonicity of a drug-delivery composition. Examples include, but are not limited to, salts, particularly sodium chloride, potassium chloride, mannitol and glycerin, or any other suitable ophthalmically acceptable tonicity adjustor. The concentration of a tonicity agent may be at least from 0.01% w/w to 7% w/w, particularly from 0.1% to 5% w/w, more particularly from 0.5% w/w to 1.5% w/w, based on the total weight of the drug-delivery composition.

According to an embodiment that can be combined with any of the other embodiments described herein, the antioxidant may include any compound or substance that is useful in reducing oxidation of any compound present in a drug-delivery composition. Suitable antioxidants include, but are not limited to sodium metabisulfite, sodium thiosulfate, acetylcysteine, butylated hydroxyanisole, and butylated hydroxytoluene.

According to an embodiment that can be combined with any of the other embodiments described herein, the drug-delivery composition is a solution comprising the intermediate composition as solute, and the liquid medium as solvent.

According to an embodiment that can be combined with any of the other embodiments described herein, the drug-delivery composition is a dispersion comprising of the intermediate composition as dispersed phase, and the liquid medium as dispersant. Further, the dispersed phase may be a colloidal dispersed phase. For the purpose of this application, the term “colloidal dispersed phase” in relation to the intermediate composition means that the dispersed phase has a particle size of from 1 μm to 500 μm, particularly of from 10 μm to 300 μm, more particularly of from 50 μm to 200 μm.

According to an embodiment that can be combined with any of the other embodiments described herein, the dispersion is a gel, a suspension or an emulsion.

According to an embodiment which can be combined with any of the other embodiments described herein, the drug-delivery composition has a viscosity which allows the drug delivery composition to adhere to the tissue (i.e. corneal surface) of the human or animal body for a sufficient time so that the pharmaceutically active compound can be released in a sustained manner (i.e. sustained drug release from the drug-delivery composition).

According to an embodiment that can be combined with any of the other embodiments described herein, the drug-delivery composition has a pH value of from 6.5 to 7.5, particularly of from 7.3 to 7.5, more particularly of from 7.3 to 7.5, typically of 7.4. A benefit of these particular pH values is that reddening and irritation of the eye is avoided.

According to an embodiment that can be combined with any of the other embodiments described herein, the drug-delivery composition has an osmolarity of from 250 mosmol/l to 350 mosmol/l, particularly of from 270 mosmol/l to 330 mosmol/l, more particularly of from 300 mosmol/l to 310 mosmol/l.

According to an embodiment that can be combined with any of the other embodiments described herein, the drug-delivery composition is sterile. This can be achieved by preparing the drug-delivery compositions under aseptic conditions or by sterilization the prepared drug-delivery compositions by sterilization techniques known to the person skilled in the art, e.g. by autoclaving, filtration, UV, gas sterilization or by a combination thereof.

According to an embodiment that can be combined with any of the other embodiments described herein the drug-delivery composition is in form of single-dose units or multi-dose units. For the purpose of this application, the term “single dose unit” intends to describe a defined portion of drug-delivery composition, which is intended to be used only once. Accordingly, term “multi-dose unit” intends to describe a defined portion of drug-delivery composition, which is intended to be used several times, e.g. at least 10 times, particularly at least 20 times.

According to an embodiment which can be combined with any of the other embodiments described herein, the drug-delivery composition is in form of liquid eye drops, oily eye drops, eye baths, eye gels, eye ointments, or eye creams.

According to an embodiment that can be combined with any of the other embodiments described herein, the drug-delivery composition is used in the treatment of eye disorders.

According to an embodiment which can be combined with any of the other embodiments described herein eye disorders are disorders selected from inflammatory eye disorders, traumatic injuries of the cornea, including perforating and non perforating corneal injuries, such as shrapnel injuries, non-infectious corneal ulcers, surgical procedures like laser vision correction, sicca syndrome, sjoegren syndrome, dry eye, or corneal transplants. According to an embodiment, which can be combined with any of the other embodiments described herein inflammatory eye disorders selected from the group consisting of conjunctivitis, blepharitis, dacryoadenitis, hordeolum, and inflammation of the tarsal, lacrimal, Meibom gland.

According to an embodiment that can be combined with any of the other embodiments described herein, the drug-delivery composition is administered to human or animal body one to five times a day, particularly two to three times a day.

The drug-delivery compositions described herein demonstrate sustained release of the pharmaceutically active compound, which can be (a) protein(s). We believe that the sustained release properties of the drug-delivery compositions are provided by the specific structure of the intermediate composition, more particularly due to the specific interaction between the pharmaceutically active compound and the hydrophilic matrix which both together form the intermediate composition. For the purpose of this application, the term “sustained release” refers to a drug release profile, wherein drug release (i.e. protein release) is maintained within a therapeutic window over a prolonged period with the objective of minimizing peak-to-trough fluctuations.

Particularly, the pharmaceutically active compound, for example the protein, is completely or to sufficient extend released from the hydrophilic matrix within a period of from 30 minutes to 5 days, particularly from 1 h to 1 day, even more particularly from 30 minutes to 12 hours, particularly of from 2 hours to 10 hours, more particularly of from 4 hours to 8 hours. This helps to reduce side effects and dosing frequency, thereby improving patient compliance. A further benefit of the drug-delivery compositions according to the present invention is that therapeutic proteins can be incorporated into a sticky matrix. Release is measured in PBS, free of further components such as enzymes, at pH 7.4.

According to an embodiment which can be combined with any of the other embodiments described herein at least 90%, typically 100% of the protein (based on the total amount of protein distributed in the intermediate composition) is released after at least 30 days, typically after 34 days, more particularly after 1 week, and further particularly after 1 day from the drug-delivery composition.

In an embodiment that can be combined with any of the other embodiments described herein, up to 60% of the protein (based on the total amount of protein distributed in the intermediate composition) is released within the first 12 hours of protein release, particularly within the first 6 hours of protein release, more particularly within the first 4 hours of protein release. Particularly, up to 70% of the protein (based on the total amount of protein distributed in the intermediate composition) is released within the first 10 hours of protein release. Typically up to 80% of the protein (based on the total amount of protein distributed in the intermediate composition) is released within the first 8 hours of protein release, particularly within the first 5 hours of protein release. In a further embodiment that can be combined with any of the other embodiments described herein, up to 40% of the protein (based on the total amount of protein distributed in the intermediate composition) is released within the first 3 hours of protein release.

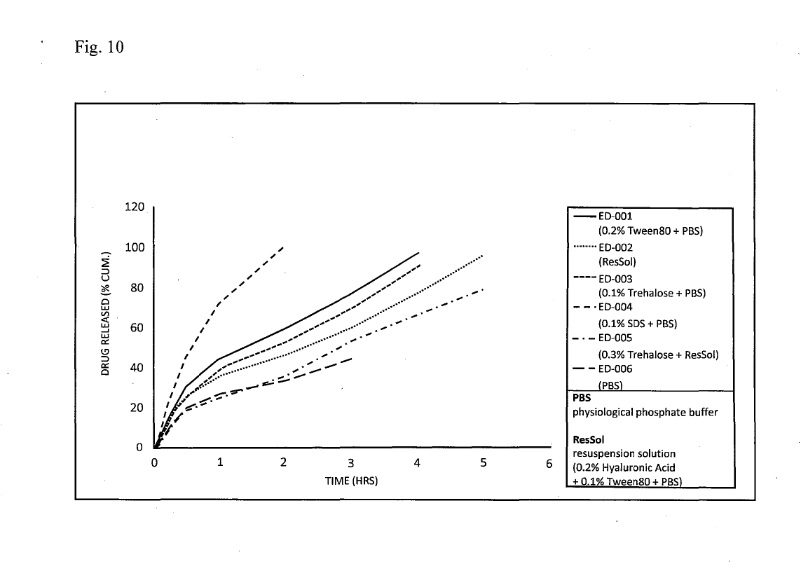

FIG. 10 illustrates the advantageous release profiles of drug delivery compositions according to embodiments.

In an embodiment which can be combined with any of the other embodiments described herein, the average release rate of the protein—defined as average mass % of protein release (based on the total mass of the protein distributed in the intermediate composition) in PBS per hour—is 5 mass % per hour, particularly 8 mass % per hour, typically 10 mass % per hour or 10 to 20 mass % per hour within the first 8 hours of protein release.

In this regard, protein release is determined into PBS of physiological ionic strength and pH at room temperature via ultraviolet spectroscopy (also modification according to Bradford). At higher resolution needs more sophisticated methods, e.g., mass spectroscopy and modifications, should be used. The PBS does not contain enzymes or surfactants.

### Intermediate Composition

For the purpose of this application, the term “intermediate composition” intends to describe a composition that is meant to be further processed in order to be transformed into a composition, which is suitable to be administered to the human or animal body for therapeutic purpose. However, in some cases, this intermediate composition is also intended to be administered directly to the human or animal body for therapeutic purpose without further processing within the scope of the underlying invention.

According to an embodiment that can be combined with any of the other embodiments described herein, the intermediate composition includes a hydrophilic matrix and a pharmaceutically active composition. Said pharmaceutically active composition comprises a pharmaceutically active compound, which particularly is a protein. However, said pharmaceutically active compound can also be any other active pharmaceutical ingredient. In a particular embodiment that can be combined with any of the other embodiments described herein, the intermediate composition consists of a hydrophilic matrix and a pharmaceutically active composition.

According to an embodiment that can be combined with any of the other embodiments described herein, said pharmaceutically active composition is dispersed in the hydrophilic matrix. Particularly, the pharmaceutically active composition is homogeneously distributed in the hydrophilic matrix. For example, the pharmaceutically active compound, for example the protein, is homogeneously distributed in the hydrophilic matrix throughout the whole intermediate composition. According to an embodiment, the hydrophilic matrix is provided in particulate form, for example in form of particles. The pharmaceutically active compound such as a protein can be homogeneously distributed throughout the hydrophilic matrix of the particles.

According to an embodiment which can be combined with any of the other embodiments described herein, the weight ratio between the hydrophilic matrix and the pharmaceutically active compound, for example the protein, is from 1:1 to 10:1, particularly 2:1 to 8:1, more particularly of from 3:1 to 6:1, typically 5:1.

According to another embodiment which can be combined with any of the other embodiments described herein, the weight ratio between the dry hydrophilic matrix and the pharmaceutically active composition is from1:1 to 1:200, particularly from 1:1 to 1:100, particularly 1:30 to 1:80, more particularly of from 1:50 to 1:70.

In a particular embodiment that can be combined with any of the other embodiments described herein, the intermediate composition is in form of particles.

According to an embodiment which can be combined with any of the other embodiments described herein, the particles (of the intermediate composition) have an average particle size of from 100 nm to 100 μm, particularly of from 500 nm to 30 μm, and more particularly of from 1 μm to 10 μm. According to an embodiment, the average particle size is from 500 nm to 5 μm. Furthermore, the particles (of the intermediate composition) can have an average particle size of from 100 nm to 500 μm, particularly of from 1 μm to 300 μm, and more particularly of from 20 μm to 200 μm. According to an embodiment, the average particle size is from 50 μm to 150 μm.

According to an embodiment which can be combined with any of the other embodiments described herein, the particles (of the intermediate composition) have an aspect ratio of from 1:1 to 50:1, particularly of from 1:1 to 10:1, particularly of from 2:1 to 10:1 more particularly of from 3:1 to 10:1. For the purpose of this application the term “aspect ratio” intends to describe the ratio of the width of the particle to its height. Hence, it may be applied to two characteristic dimensions (such as width and height) of a three-dimensional shape (such as a particle).

According to an embodiment which can be combined with any of the other embodiments described herein, the particles (of the intermediate composition) are flat shaped and non-spherical, i.e. have a thickness less than their length or width, for example each of the length and width is at least twice the height, or at least three-times the height of the particles.

According to an embodiment that can be combined with any of the other embodiments described herein, the particles (of the intermediate composition) exhibit a flat shape. In this respect, in idealized form, a particle has 3 dimensions x, y and z referring to the length, width and height. Each dimension has a maximum diameter (due to the irregular shape of the particle dimensions). A particle having a flat shape is defined by the maximum diameter of one dimension (e.g. dimension z: height) being at least twice as small as the smaller one of the maximum diameters of the two other dimensions (i.e. x and y). Particularly, the maximum diameter of one dimension (e.g. dimension z: height) is at least four times smaller than the smaller one of the maximum diameters of the two other dimensions (i.e. x and y). This particular flat shape of the particles of the intermediate composition leads to the effect that a maximum contact surface (between the intermediate composition and the corneal surface) is achieved, which is beneficial for an effective drug delivery to the eye.

A flat shape of the particles is believed to increase the contact area between the particles and the corneal surface which results in a prolonged availability of the particles on the eye in comparison to, for example, spherical particles. In addition to the flat particle shape the duration of particle adhesion is increased by covalent coupling of the particle-constituting macromolecules. The covalent coupling of the particle components is reducing the probability of whole particle release from target surface. The prolonged availability ensures that the reservoir provided by the particles for the pharmaceutically active compound, particularly for proteins, remains in contact with the eye for a longer time which contributes, together with the sustained release properties provided by the hydrophilic matrix, to an improved and prolonged availability of the pharmaceutically active compound. Hence, the pharmaceutically active compound can be delivered over a longer period of time in comparison to, for example, conventional drug delivery compositions, which do not exhibit the above described physical delayed and sustained effect of improved adhesiveness provided by the flat shape of the particles.

According to an embodiment that can be combined with any of the other embodiments described herein, the intermediate composition is sticky. For the purpose of this application, the term “sticky” intends to describe the tendency of the intermediate composition (particularly the intermediate composition in form of particles) to adhere to the cornea or other surfaces when being administered in form of the drug-delivery composition. Thereby, the forces that cause this tendency may fall into the categories of mechanical adhesion (e.g. interlocking), chemical adhesion (e.g. ionic, covalent, or hydrogen bonds), dispersive adhesion (e.g. van der Waals forces), and diffusive adhesion. This sticky character of the intermediate composition is based on the specific structure of the intermediate composition and leads to the effect that the intermediate composition (comprised in the drug-delivery composition) remains on the corneal or other surfaces sufficiently long enough to release at least 10%, particularly at least 50%, further particularly at least 70% of the total amount of pharmaceutically active compound (i.e. protein) to the eye (cornea) of the human or animal.

### Hydrophilic Matrix

For the purpose of this application, the term “hydrophilic matrix” intends to describe a macromolecular polymer system which has polar functional groups and swells in a restricted manner, restriction caused by covalent cross-linking when mixed with the aqueous solution.

According to an embodiment that can be combined with any of the other embodiments described herein, the hydrophilic matrix comprises particles. In a particular embodiment that can be combined with any of the other embodiments described herein, the hydrophilic matrix is in form of particles as described above. The main component of the particles can be the hydrophilic matrix. For example, the particles can essentially consist of the hydrophilic matrix and the pharmaceutically active composition as described above.

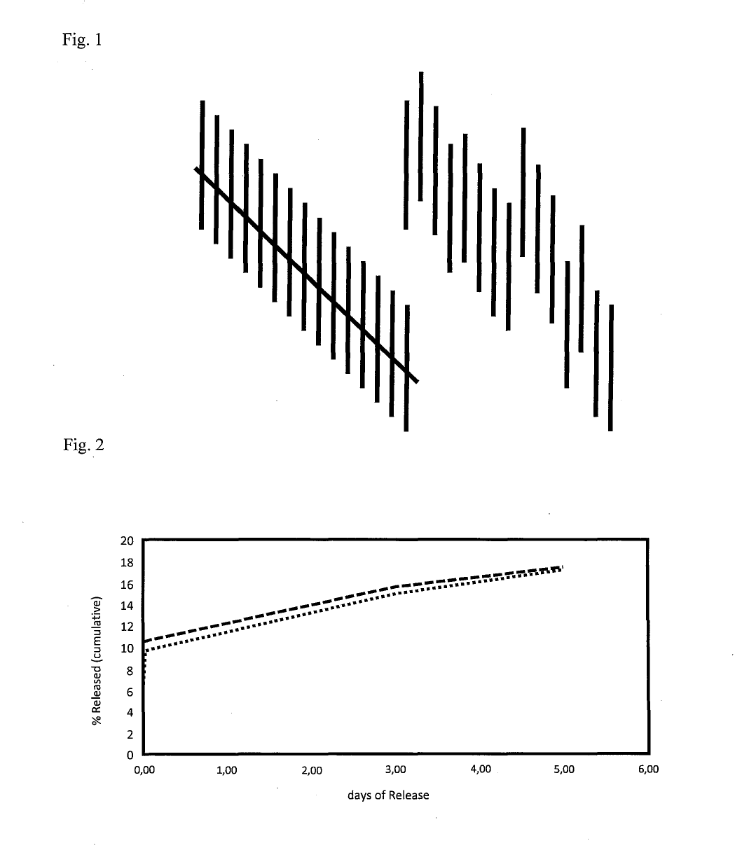

The hydrophilic matrix is typically in form of particulate units rather than in form of a solution of individual macromolecules. This approach takes into account colloid chemical basic rules of dependence of adhesion strength on size of interaction area and statistics of multi-site adhesive contact points, their persistence and stability. We believe that a flat particle comprising a number n (typically a large number) of connected or cross-linked molecules is more adhesive than the same number of molecules in an unconnected manner. This particle adhesion promoting effect is even amplified by the statistics of bound and un-bound states of molecular focal point contacts to a substrate (cornea) surface. The probability that all (or majority) of contacts are broken simultaneously is rather small and as such is promoting the bound versus un-bound status (presumed that interaction energy is in the order of a few kT). It is intuitively clear that the left structure of the scheme in FIG. 1 would adhere more stable to a cornea surface than the right structure of un-connected “molecules” as illustrated in FIG. 1.

According to an embodiment which can be combined with any of the other embodiments described herein the particles have an average particle size of from 100 nm to 500 μm, particularly of from 1 μm to 300 μm, and more particularly of from 50 μm to 150 μm.

According to an embodiment which can be combined with any of the other embodiments described herein the particles have an average particle size of from 100 nm to 50 μm, particularly of from 500 nm to 30 μm, and more particularly of from 1 μm to 10 μm.

According to an embodiment that can be combined with any of the other embodiments described herein the particles are biodegradable. For the purpose of this application, the term “biodegradable” intends to describe a material that is degradable within the animal or human body by enzymatic of cellular processes.

According to an embodiment that can be combined with any of the other embodiments described herein the particles are biocompatible. For the purpose of this application, the term “biocompatible” intends to describe the nature of a material (i.e. biomaterial) to cause an acceptable host response when being in contact with the host (i.e. animal or human body).

According to an embodiment that can be combined with any of the other embodiments described herein the particles essentially exhibit pores.

According to an embodiment that can be combined with any of the other embodiments described herein the pore size of the particles is small enough to ensure retention of the proteins to retard their release. For example, the pore size can approximately corresponds to the radius of gyration of the proteins.

According to an embodiment that can be combined with any of the other embodiments described herein the particles have an aspect ratio of from 1:1 to 50:1, particularly of from 1:1 to 20:1, particularly of from 2:1 to 20:1, particularly of from 3:1 to 20:1, particularly of from 2:1 to 10:1, more particularly of from 3:1 to 10:1.

According to an embodiment that can be combined with any of the other embodiments described herein the particles comprise at least one cross-linked polymer. For the purpose of this application, the term “polymer” is used as a synonym for the term “macromolecular compound”.

For the purpose of this application, the term “cross-linked” intends to describe covalent bonds that link one polymer chain of the macromolecular compound to one another. As a consequence, the polymers' physical properties are changed. According to an embodiment, the cross-linking means covalently cross-linking.

According to an embodiment which can be combined with any of the other embodiments described herein the cross-linked macromolecular compound is at least one polymer having a molecular weight (in the non cross-linked form) of at least 10000 Da, particularly of from 50000 Da to 4 MDa, more particularly of from 100000 Da to 3 MDa.

According to an embodiment that can be combined with any of the other embodiments described herein the cross-linked macromolecular compound (i.e. cross-linked polymer) is a naturally occurring compound (i.e. polymer).

According to an embodiment that can be combined with any of the other embodiments described herein the cross-linked macromolecular compound (i.e. cross-linked polymer) is a synthetic compound (i.e. polymer).

According to an embodiment that can be combined with any of the other embodiments described herein the cross-linked macromolecular compound (i.e. cross-linked polymer) is selected from hyaluronic acid, fibrin, polyvinyl alcohol (PVA). Further suitable compounds are polyvinylpyrrolidone (PVP), gelatin, collagen, alginate, starch, cellulose, chitosan, carboxymethylcellulose, cellulose derivatives, pectin, gum arabic, carrageenan, albumin, fibrinogen, synthetic polyelectrolytes, polyethylenimine, acacia gum, xanthan gum, agar agar, polyvinylalcohol, borax, polyacrylic acids including derivatives, protaminsulfate, casein, and derivatives thereof. According to an embodiment, inorganic polymers such as clay and silica can also be used for the hydrophilic matrix. Furthermore, polyampholytes can be used as a polymer component. According to an embodiment, a polymer from the group of biopolymers is used. According to an embodiment, a polymer from the group of hydrogel forming substances such as gelatin is used. According to an embodiment, a polymer from the group of polyelectrolyte complex forming substances is used. Such substances typically include two components of opposite charge selected from two polyelectrolytes of opposite charge and a polyelectrolyte and a small ion of opposite charge such as alginate and calcium. According to an embodiment, a polymer from the group of polyampholytes is used. According to an embodiment, a polymer from the group of inorganic gel forming substances is used. According to an embodiment, a polymer from the compound class of glycosaminoglycans is used. In particular, those glycosaminoglycans are selected from the group consisting of hyaluronic acid, heparin sulfate, chondroitin sulfate, dermatan sulfate and keratin sulfate.

According to an embodiment that can be combined with any of the other embodiments described herein, the dry hydrophilic matrix (particles) is contained in the drug delivery composition in a concentration of from 0.1% w/w to 20% w/w, particularly of from 0.1% w/w to 10% w/w, more particularly of from 0.1% w/w to 5% w/w, further particularly of from 0.1% w/w to 3% w/w based on the total weight of the drug-delivery composition. Furthermore, the dry hydrophilic matrix (particles) is contained in the drug delivery composition in a concentration of from 0.3% w/w to 10% w/w, particularly of from 0.3% w/w to 5% w/w, more particularly of from 0.3% w/w to 2% w/w.

### Pharmaceutically Active Composition

For the purpose of this application, the term “pharmaceutically active composition” intends to describe any pharmaceutical dosage form known to those skilled in the art, which comprises a pharmaceutical active compound, for example a protein. For instance, these dosage forms comprise dispersions such as suspensions or emulsions, or solutions.

According to an embodiment that can be combined with any of the other embodiments described herein, the pharmaceutically active composition comprises a pharmaceutically active compound which is a protein. However, said pharmaceutically active compound may also be any other active pharmaceutical ingredient known to the person skilled in the art.

According to an embodiment that can be combined with any of the other embodiments described herein, the pharmaceutical active compound is contained in the pharmaceutically active composition in a concentration of from 1 mg/ml to 100 mg/ml, particularly of from 10 mg/ml to 50 mg/ml, particularly of from 15 mg/ml to 40 mg/ml, more particularly of from 20 mg/ml to 30 mg/ml.

According to an embodiment that can be combined with any of the other embodiments described herein, the pharmaceutically active composition further comprises a liquid component. In a particular embodiment that can be combined with any of the other embodiments described herein, the pharmaceutically active composition consists of a protein and a solvent.

According to an embodiment that can be combined with any of the other embodiments described herein, the liquid component is selected from hydrophilic solvents, lipophilic solvents and solubilizers, or in any combination of two or more kinds thereof.

According to an embodiment which can be combined with any of the other embodiments described herein, the hydrophilic solvent is selected from the group consisting of water, ethanol, glycerol, 1,2-propylene-glycol, low-molecular polyethylene-glycoles (PEG 200, PEG 300, PEG 400), N-methyl-2-pyrrolidone (NMP, Pharmasolve), dimethylacetamide, dimethyl sulfoxide (DMSO), isopropanol, benzyl alcohol and tensides (such as Cremophor EL, Cremophor RH 60, Polysorbat 80 and Solutol HS 15).

According to an embodiment which can be combined with any of the other embodiments described herein, the lipophilic solvent is selected from the group consisting of fatty acid esters, isopropylmyristate, -palmitate, -stearate; oleic acid oleyl ester, liquid triglycerides such as Glyceroltriacetat or oils. Said oils are selected from the group consisting of castor oil, clove oil, cassia oil, almond oil, corn oil, arachis oil, cottonseed oil, safflower oil, maize oil, linseed oil, rapeseed oil, soybean oil, caraway oil, rosemary oil, peanut oil, peppermint oil, sunflower oil, eucalyptus oil, olive oil, mentha oil, peppermint oil, eucalyptus oil, bergamot oil, anise oil, fennel oil, or rose oil.

According to an embodiment which can be combined with any of the other embodiments described herein, the solubilizer is selected from the group consisting of polyoxyethylene-polyoxypropylene (POE-POP) block copolymers, cyclodextrins (e.g. (3-cyclodextrin, y-cyclodextrin), cyclodextrin derivatives (e.g. sulfobutyl or hydroxypropyl ethers), bile acids, bile acid derivatives, sterol derivatives, alcohols, particularly, fatty alcohols and fatty alcohol derivatives, acids, particularly fatty acids and fatty acid derivatives and tocol derivatives.

According to an embodiment that can be combined with any of the other embodiments described herein, the liquid component differs from the liquid medium described above.

According to an embodiment that can be combined with any of the other embodiments described herein, the liquid component is water, typically aqua ad injectabilia.

According to an embodiment that can be combined with any of the other embodiments described herein, the pharmaceutically active composition is a solution comprising the pharmaceutically active compound as solute, and a liquid component as solvent.

According to an embodiment that can be combined with any of the other embodiments described herein, the pharmaceutically active composition is a dispersion comprising the pharmaceutically active compound as dispersed phase, and the liquid component as dispersant.

According to an embodiment that can be combined with any of the other embodiments described herein, the dispersed phase is a colloidal dispersed phase.

For the purpose of this application, the term “colloidal dispersed phase”, as related to the pharmaceutically active composition, is intended to describe that the dispersed phase has a particle size of from 1 nm to 1 μm, particularly of from 10 nm to 800 nm, more particularly of from 50 nm to 500 nm.

According to an embodiment that can be combined with any of the other embodiments described herein, the dispersion is a gel, a suspension or an emulsion.

According to an embodiment that can be combined with any of the other embodiments described herein, the pharmaceutically active composition has a pH value from 6.0 to 8.0, particularly from 6.5 to 7.5, more particularly from 7.3 to 7.5.

According to an embodiment that can be combined with any of the other embodiments described herein, the pharmaceutically active composition has an osmolarity of from 250 mosmol/l to 350 mosmol/l, particularly of from 270 mosmol/l to 330 mosmol/l, more particularly of from 300 mosmol/l to 310 mosmol/l.

According to an embodiment that can be combined with any of the other embodiments described herein, pharmaceutically active composition comprises at least one excipient.

According to an embodiment which can be combined with any of the other embodiments described herein, the excipient comprised in the pharmaceutically active composition is selected from the group consisting of monosaccharides, disaccharides, oligosaccharides, polysaccharides like hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof. An advantage thereof consists in further modifying release characteristics of the drug-delivery composition.

According to an embodiment a drug delivery composition is manufactured, wherein the dissolved pharmaceutically active composition comprises at least a pharmaceutically active compound without any excipients.

### Pharmaceutically Active Compound

For the purpose of this application, the term “pharmaceutically active compound” intends to describe a pharmaceutical drug, which is biologically active and is referred to hereinafter as active pharmaceutical ingredient (API).

According to an embodiment that can be combined with any of the other embodiments described herein, the pharmaceutically active compound is a protein. However, said pharmaceutically active compound may also be any other active pharmaceutical ingredient known to the person skilled in the art. Particularly, the pharmaceutically active compound is selected from the group consisting of nucleic acids, aptamers, spiegelmers

According to an embodiment that can be combined with any of the other embodiments described herein, the pharmaceutically active compound is selected from the group consisting of: immunoglobulins, fragments or fractions of immunoglobulins, synthetic substances mimicking immunoglobulins or synthetic, semisynthetic or biosynthetic fragments or fractions thereof, chimeric, humanized or human monoclonal antibodies, Fab fragments, fusion proteins or receptor antagonists (e.g., anti TNF alpha, Interleukin-1, Interleukin-6 etc.), antiangiogenic compounds (e.g., anti-VEGF, anti-PDGF etc.), costimulatory signal inhibitors (e.g. abatacept, alefacept), intracellular signaling inhibitors (e.g. JAK1,3 and SYK inhibitors) or other compounds targeting cellular signaling mechanisms or surface antigens on B and T cells (eg anti CD4, 20, 52 etc), peptides having a molecular mass equal to or higher than 3 kDa, ribonucleic acids (RNA), desoxyribonucleic acids (DNA), plasmids, peptide nucleic acids (PNA), steroids, corticosteroids, an adrenocorticostatic, an antibiotic, an antidepressant or other mood stabilizers, an antimycotic, a [beta]-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an antiinflammatory drug, an anticholinergic, an antihistaminic, an antihypertensive, an antihypotensive, an anticoagulant, an antiseptic, an anti-hemorrhagic, an anti-myasthenic, an antiphlogistic, an antipyretic, a beta-receptor antagonist, a calcium channel antagonist, a cell, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a glucocorticoid, a growth factor, a haemostatic, a hormone and its synthetic or biosynthetic analogue, an immunosuppressant, an immunostimulant, a mitogen, a physiological or pharmacological inhibitor of mitogens, a mineralcorticoid, a muscle relaxant, a narcotic, a neurotransmitter, a precursor of a neurotransmitter, an oligonucleotide, a peptide, a (para)-sympathicomimetic, a (para)-sympatholytic, a protein, a sedating agent, a spasmolytic, a vasoconstrictor, a vasodilatator, a vector, a virus, a virus-like particle, a virustatic, a wound-healing substance, or in any combination of two or more kinds thereof.

According to an embodiment which can be combined with any of the other embodiments described herein the protein comprised in the pharmaceutically active composition has a molecular weight of from 5 kDalton to 700 kDalton, particularly of form 10 kDalton to 600 kDalton, more particularly of from 15 kDalton to 500 kDalton.

According to an embodiment that can be combined with any of the other embodiments described herein, the protein comprised in the pharmaceutically active composition is selected from the group consisting of therapeutic proteins such as immunoglobulins, fragments or fractions of immunoglobulins, synthetic substances mimicking immunoglobulins or synthetic, semisynthetic or biosynthetic fragments or fractions thereof, chimeric, humanized or human monoclonal antibodies, Fab fragments, fusion proteins or receptor antagonists (e.g., anti TNF alpha, Interleukin-1, Interleukin-6 etc.), antiangiogenic compounds (e.g., anti-VEGF, anti-PDGF etc.), costimulatory signal inhibitors (e.g. abatacept, alefacept), intracellular signaling inhibitors (e.g JAK1,3 and SYK inhibitors) or other compounds targeting cellular signaling mechanisms or surface antigens on B and T cells (eg anti CD4, 20, 52 etc), proteins which are used as an antibiotic, an antidepressant or other mood stabilizers, an antimycotic, a [beta]-adrenolytic, an androgen or antiandrogen, an antianemic, an anabolic, an anaesthetic, an analeptic, an antiallergic, an antiarrhythmic, an antiarterosclerotic, an antibiotic, an antifibrinolytic, an anticonvulsive, an antiinflammatory drug, an anticholinergic, an antihistaminic, an antihypertensive, an antihypotensive, an anticoagulant, an antiseptic, an anti-hemorrhagic, an anti-myasthenic, an antiphlogistic, an antipyretic, a beta-receptor antagonist, a calcium channel antagonist, a cell differentiation factor, a chemokine, a chemotherapeutic, a coenzyme, a cytotoxic agent, a prodrug of a cytotoxic agent, a cytostatic, an enzyme and its synthetic or biosynthetic analogue, a growth factor, a haemostatic, a hormone and its synthetic or biosynthetic analogue, an immunosuppressant, an immunostimulant, a mitogen, a physiological or pharmacological inhibitor of mitogens, a muscle relaxant, a narcotic, a neurotransmitter, a precursor of a neurotransmitter, a (para)-sympathicomimetic, a (para)-sympatholytic, a protein, a sedating agent, a spasmolytic, a vasoconstrictor, a vasodilatator, a virustatic, a wound-healing substance, or in any combination of two or more kinds thereof.

According to an embodiment, the therapeutic protein is an antibody, an enzyme, a chemokine or an interferon.

According to an embodiment that can be combined with any of the other embodiments described herein, the protein comprised in the pharmaceutically active composition is an interleukin-1 (Il-1) receptor antagonist (IL-1ra), a monoclonal antibody against IL-1 (IL-1 Mab), IL-1 trap or anti IL-1 fusion protein. Preferably, the interleukin-1 (Il-1) receptor antagonist (IL-1ra) is an interleukin-1 (IL-1) alpha or beta antagonist,

According to a particular preferred embodiment that can be combined with any of the other embodiments described herein, the protein comprised in the pharmaceutically active composition is an interleukin-1 (IL-1) receptor antagonist (IL-1ra), preferably an interleukin-1 (IL-1) alpha or beta antagonist. According to another particular preferred embodiment that can be combined with any of the other embodiments described herein, the protein comprised in the pharmaceutically active composition is a monoclonal antibody against IL-1.

IL-1 is a cytokine that is released by white blood cells and has specific effects on cell-cell interactions, communication, and behavior of other cells. Wound healing process following any corneal injury, trauma, surgery or in corneal transplantation is generally initiated by a release of various proteins that are involved in cell-to-cell signaling. Particularly, wound healing processes have been shown to be highly dependent on one key cytokine, i.e. IL-1. IL-1 mediates and facilitates the immune response of the corneal healing process. IL-1 trap is a dimeric fusion protein consisting of the ligand-binding domains of the extracellular portions of the human interleukin-1 receptor component (IL-1R1) and IL-1 receptor accessory protein (IL-1RAcP) linked to a monoclonal antibody against IL-11 (IL-1 Mab).

According to an embodiment, the above mentioned proteins are particularly used for topical eye treatments, for example to promote corneal wound healing after eye injury or LASIK treatment.

According to an embodiment that can be combined with any of the other embodiments described herein the protein comprised in the pharmaceutically active compound is a competitive receptor or direct cytokine antagonist.

According to an embodiment that can be combined with any of the other embodiments described herein the protein comprised in the pharmaceutically active compound is a naturally occurring protein.

According to an embodiment which can be combined with any of the other embodiments described herein the protein is encoded in humans by the IL1RN gene.

According to an embodiment which can be combined with any of the other embodiments described herein the protein is among other an IL-1ra. IL-1ra is a biologic agent (protein) that blocks IL-1.

According to an embodiment that can be combined with any of the other embodiments described herein the protein is the pharmaceutically active compound.

According to an embodiment that can be combined with any of the other embodiments described herein, the pharmaceutical active compound is contained in the drug delivery composition in a concentration of from 1 mg/ml to 100 mg/ml, particularly from 2 mg/ml to 80 mg/ml, more particularly from 3 mg/ml to 60 mg/ml, typically from 4 mg/ml to 55 mg/ml, based on the total weight of the drug-delivery composition.

According to an embodiment that can be combined with any of the other embodiments described herein, the pharmaceutical active compound is contained in the drug delivery composition in a concentration of from 0.01% w/w to 20% w/w, particularly of from 0.1% w/w to 10% w/w, more particularly of from 0.5% w/w to 7% w/w, based on the total weight of the drug-delivery composition.

### Method for Manufacturing a Drug-Delivery Composition

According to an embodiment, a method for manufacturing a drug-delivery composition includes providing a hydrophilic matrix and providing a pharmaceutically active composition which includes a protein. The hydrophilic matrix is provided in form of particles substantially composed of at least one cross-linked polymer. Said method further includes mixing the hydrophilic matrix and the protein in a weight ratio from 1:1 to 10:1 to form an intermediate composition.

According to an embodiment, a method for manufacturing a drug-delivery composition includes providing a liquid film, for example, an aqueous film of a non-cross-linked polymer having a molecular weight of at least 10000 Da. The liquid film of the non-cross-linked polymer is at least partially dried to obtain an at least partially dried film of the non-cross-linked polymer having a moisture content of less than 30%, particularly less than 10%. A cross-linking agent is added to the at least partially dried film of the non-cross-linked polymer to cross-link the polymer. The film of the cross-linked polymer is dried at an elevated temperature to obtain a dried film of the non-cross-linked polymer. The dried film of the cross-linked polymer is broken to particles by a mechanical process to obtain particles having an aspect ratio of 1:1 to 50:1, particularly of 2:1 to 50:1, and an average particle size of 100 nm to 100 μm.

### Process of Providing a Hydrophilic Matrix

A benefit of such a manufacturing method can be seen in achieving a sustained release formulation for pharmaceutically active ingredients with improved release characteristics. In particular, the method allows preparing drug-delivery compositions for sustained release of ingredients characterized by a specific biological activity which otherwise might decrease or even terminate.

According to an embodiment, which can be combined with any of the other embodiments described herein, providing a hydrophilic matrix comprises, in a first step, providing a film of a macromolecular compound (i.e. polymer).

According to an embodiment, which can be combined with any of the other embodiments described herein, providing a film of a macromolecular compound that is intended to describe the provision of a film comprising a macromolecular compound (i.e. polymer) which is not cross-linked (i.e. in a non-cross-linked form) in a concentration of from 0.1 mass % to 10 mass %, particularly of from 1 mass % to 5 mass %, more particularly of from 1.5 mass % to 3 mass %, typically 2 mass %, based on the total mass of the film.

According to an embodiment that can be combined with any of the other embodiments described herein providing a hydrophilic matrix further comprises, in a second step, a first drying step in order to obtain an at least partially dried film. The first drying step can be performed at an elevated temperature of from 30° C. to 70° C., particularly of from 35° C. to 60° C., more particularly of from 35° C. to 60° C., typically at 50° C. According to an embodiment that can be combined with any of the other embodiments described herein the first drying step is carried out for 0.5 hour to 5 hours, particularly for 1 hour to 3 hours, typically for 2 hours. The resulting dried film of non-cross-linked macromolecular compound does not need to be completely dried but it is sufficient that it is substantially dry. For example, the moisture content can be about 30% or less, particularly 10% or less. The drying leads to a reduction of the film thickness and the formation of a compact and dense film For example, the thickness can reduce from the initial thickness of the liquid film to the at least partially dry film by a factor of about 10. As an example, an initially 1 mm thick liquid film can reduce to an about 100 μm thick partially or completely dry film.

According to an embodiment, which can be combined with any of the other embodiments described herein, providing a hydrophilic matrix further comprises, in a third step, a step of cross-linking the macromolecular compound of said dried film. The step of crosslinking the macromolecular compound (i.e. polymer) of the film comprises, for example, adding 3 M HCl and 50% glutaraldehyde solution (mixture ratio: 1 to 1) to the film comprising the macromolecular compound (i.e. polymer) in a non cross-linked form. This initiates crosslinking of said macromolecular compound (i.e. polymer). According to an embodiment that can be combined with any of the other embodiments described herein the step of crosslinking the macromolecular compound (i.e. polymer) is carried out for 0.5 hour to 7 hours, particularly for 1 hour to 5 hours, typically for 3 hours. Alternatively, the step of crosslinking the macromolecular compound (i.e. polymer) is carried out for 5 or 10 minutes to 1 hour, particularly for 8 minutes to 40 minutes, typically for 10 minutes.

Crosslinking is not restricted to the given example, other crosslinking reagents and protocols can be applied.

When adding the cross-linking agent, which can be provided as solution, the dry film can undergo swelling. The swelling is, however, restricted due to the onset of the cross-linking. Since the cross-linking starts with the substantially dry and dense film, a dense cross-linked film is obtained, which is typically denser than a cross-linked film manufactured from a liquid film. It is believed that the density of the cross-linked film influences the release-properties as observed.

The step of cross-linking the macromolecular compound may further comprise a washing process to remove all not-reacting cross-linker. This is achieved by extensive washing in aqua ad injectabilia. After cross-linking, one or more washing processes can be carried out to remove the one or more cross-linking agents and possibly non-cross-linked macromolecular compound.

According to an embodiment, which can be combined with any of the other embodiments described herein, the step of providing a hydrophilic matrix further comprises, in a fourth step, a second drying step to obtain a dried cross-linked material. The second drying step is performed by an elevated temperature of from 30° C. to 70° C., particularly of from 35° C. to 60° C., more particularly of from 35° C. to 60° C., typically at 50° C. According to an embodiment that can be combined with any of the other embodiments described herein the second drying step is carried out for 0.5 hour to 10 hours, particularly for 1 hour to 5 hours, typically for 2 hours.

According to an embodiment, which can be combined with any of the other embodiments described herein, the step of providing a hydrophilic matrix further comprises, in a fifth step, a step of breaking up the material obtained from the fourth step to small pieces of below 100 μm. The step of breaking up comprises any mechanical process known to those skilled in the art for reducing the size of the material obtained from the fourth step. Such mechanical process includes comminuting material by any cutting means, such as scissors, milling tools, drilling tools, lathe tools or abrasive tools. The resulting particles typically have a flat shape as the particles were prepared from the dry cross-linked film.

According to an embodiment that can be combined with any of the other embodiments described herein the step of providing a hydrophilic matrix further comprises, in a sixth step, a milling step. The milling step comprises any milling process known to those skilled in the art. Particularly, the term “milling step” intends to describe any process of grinding, cutting, pressing, or crushing of the material obtained from the fifth step to small pieces in order to further reduce the size of said material. According to an embodiment that can be combined with any of the other embodiments described herein the milling step is carried out in a ball mill (e.g. Pulverisette 23, Fritsch GmbH, Germany).

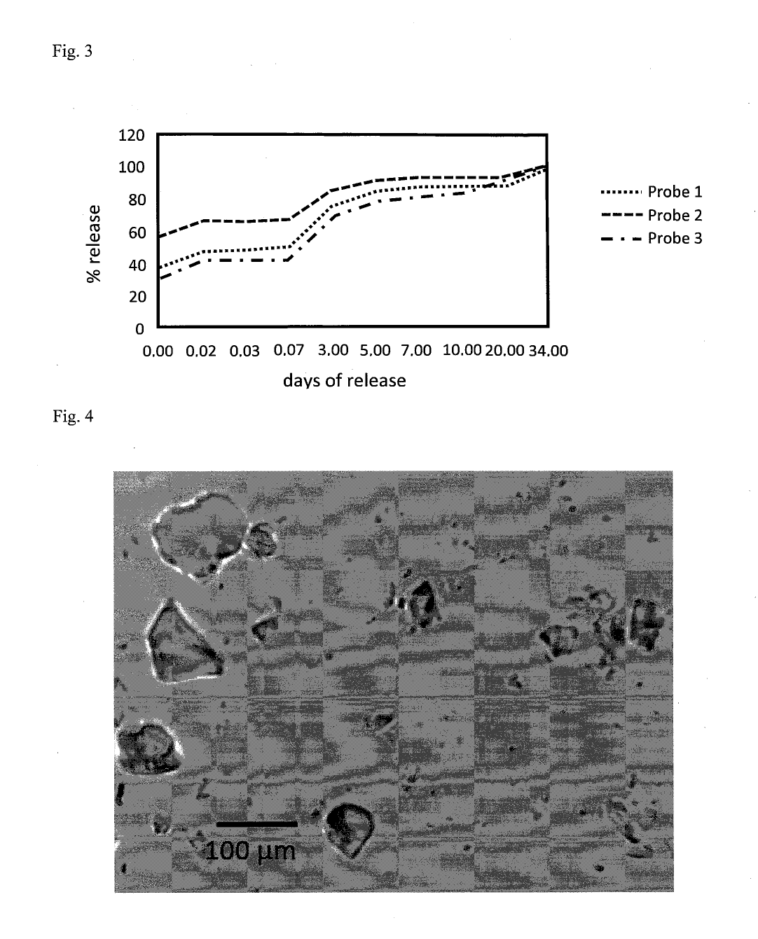

According to an embodiment that can be combined with any of the other embodiments described herein the step of providing a hydrophilic matrix further comprises, in a seventh step, a sieving step. The sieving step comprises any sieving process known to those skilled in the art. Particularly, the term “sieving step” intends to describe any process of for separating particles of different sizes. This may be achieved by fractionated sieving with the help of a stack of two or more sieves having different pore sizes. According to an embodiment which can be combined with any of the other embodiments described herein the sieving step is carried out using a metal sieve having a pore size of 30 μm to 150 μm, typically 100 μm, or of 30 μm to 60 μm, typically 50 μm. Typical examples of hydrophilic matrix particles obtained are given in FIG. 3.

Depending on the processes used, the obtained particles can be spherical or flat shaped. Flat shaped particles are particularly obtainable when starting from the above described film making processes. For example, particles with an aspect ratio of 1.1:1 to 50:1, particularly 2:1 to 50:1, particularly 3:1 to 50:1 can be obtained.

### Process of Providing a Pharmaceutically Active Composition

According to an embodiment which can be combined with any of the other embodiments described herein providing a pharmaceutically active composition comprises mixing of at least a pharmaceutical active compound (AIP) and a liquid component. According to an embodiment which can be combined with any of the other embodiments described herein mixing is carried out at a temperature of from 18° C. to 40° C., particularly of from 20° C. to 30° C., typically at 25° C.

### Process of Mixing the Hydrophilic Matrix and the Pharmaceutically Active Composition

According to an embodiment which can be combined with any of the other embodiments described herein the step of mixing the hydrophilic matrix, particularly in form of particles, and the pharmaceutically active compound (i.e. protein) is carried out in a weight ratio of from 20:1 to 1:1, particularly of from 15:1 to 2:1, more particularly of from 10:1 to 3:1, typically of 10:1, or in a weight ratio of from 10:1 to 1:1, particularly of from 8:1 to 2:1, more particularly of from 6:1 to 3:1, typically of 5:1 to form an intermediate composition. The above ratios are defined between the weight of the dry hydrophilic matrix and the weight of the pharmaceutically active compound. A benefit of the specific weight ratio between the hydrophilic matrix and the pharmaceutically active compound or composition is that the pharmaceutically active composition is substantially completely taken up by the hydrophilic matrix. In other words, the pharmaceutically active composition is homogeneously dispersed in the hydrophilic matrix.

According to an embodiment that can be combined with any of the other embodiments described herein, no heating is used within the step of mixing the hydrophilic matrix and the pharmaceutically active composition.

In particular the intermediate composition is kept throughout the mechanical treatment in a non-molten state.

According to an embodiment that can be combined with any of the other embodiments described herein, active cooling is used in order to keep the intermediate composition in a non-molten state. This approach prevents self-organization processes to occur.

According to an embodiment that can be combined with any of the other embodiments described herein the temperature of the intermediate composition can be kept below a certain temperature value by cooling. Advantageously, that allows protecting susceptible biologically active substances such as proteins from denaturation, for instance by keeping the temperature of the mixture below 30° C., below 37° C., below 45° C., below 50° C., or especially below 60° C.

According to an embodiment which can be combined with any of the other embodiments described herein providing a hydrophilic matrix further comprises, in a fourth step, forming the elastic body obtained into a plate.

### Process of Mixing Intermediate Composition and Liquid Medium

According to an embodiment which can be combined with any of the other embodiments described herein the method for manufacturing a drug-delivery composition further includes providing a liquid medium as described herein and mixing the intermediate composition and the liquid medium in a weight ratio of from 0.1:100 to 10:100, particularly 1:100 to 10:100, more particularly of from 2:100 to 10:100 to form a mixture.

According to an embodiment which can be combined with any of the other embodiments described herein the method for manufacturing a drug-delivery composition further comprises the step of adjusting the mixture to a pH value of from 6.0 to 8.0, particularly of from 6.5 to 7.5, more particularly of from 7.3 to 7.5 by adding buffer agents as described herein.

According to an embodiment which can be combined with any of the other embodiments described herein the method for manufacturing a drug-delivery composition further comprises the step of adjusting the mixture to an osmolarity of from 250 mosmol/l to 350 mosmol/l, particularly of from 270 mosmol/l to 330 mosmol/l, more particularly of from 300 mosmol/l to 310 mosmol/l by adding tonicity agents as described herein.

### Use

According to an embodiment that can be combined with any of the other embodiments described herein, the drug-delivery composition can be used in ophthalmic and other formulations.

According to an embodiment that can be combined with any of the other embodiments described herein ophthalmic compositions are compositions selected from the group consisting of semisolid or liquid compositions such as ointments, creams, gels, solutions.

According to an embodiment which can be combined with other embodiments, the drug-delivery composition can be used in topical applications, for example transdermal, for treatment of inflammations, immunological diseases, auto-immune diseases such as arthritis, to name few.

According to an embodiment, the drug-delivery composition can be injected.

### Method for Delivery

According to an embodiment which can be combined with any of the other embodiments described herein, a method for delivery of the drug-delivery composition is provided, comprising the steps of providing a drug-delivery composition and applying the drug-delivery composition to/into a human or animal body.

According to an embodiment which can be combined with any of the other embodiments described herein, the step of applying the drug-delivery composition to a human or animal body comprises applying the drug-delivery composition topically to a human or animal body, preferably to one or both eyes of the human being or animal.

According to an embodiment which can be combined with any of the other embodiments described herein, the step of applying the drug-delivery composition topically to a human or animal body comprises dropping the drug-delivery composition onto one or both eyes of the human being or animal.

According to an embodiment which can be combined with any of the other embodiments described herein, the step of applying the drug-delivery composition into a human or animal body comprises injecting the composition into a human or animal body.

According to an embodiment that can be combined with any of the other embodiments described herein, injecting means subcutaneous injecting, intramuscular injecting, intraperitoneal injecting, intravitreal, subconjunctial, intraarticular injecting.

According to an embodiment that can be combined with any of the other embodiments described herein, the hydrophilic matrix as described herein can be used as therapeutic protein release system in ophthalmic compositions.

According to an embodiment, the drug-delivery composition is applied drop-wise to the eye of a patient. For example, the drops can be placed under the upper eyelid of the patient.

### PREPARATION EXAMPLE

According to an embodiment, a polymer solution containing a not cross-linked polymer of at least 10000 Da is provided. With the aid of a cross-linking agent, the polymer is covalently cross-linked. The cross-linking can result in the formation of a solid body, which keeps its shape. The cross-linked polymer body is then physically broken to form particles, particularly flat particles. As an example, the polymer solution can be poured onto a supporting substrate to form a thin foil with a predetermined thickness of, for example, few micrometers.

The polymer solution can be dried prior to cross-linking, for example in air at an elevated temperature of at least about 40° C., typically about 50° C. or about 60° C. The upper limit should be below 100° C. The drying can also be carried out even at room temperature or even by complete or partial lyophilization. Basically, any suitable process can be used to at least partially remove the solvent from the polymer solution as described above.

The dried polymer film can then be cross-linked, for example by adding a solution containing a suitable cross-linking agent or a mixture of cross-linking agents followed by an optional washing process to remove not cross-linked polymer and/or cross-linking agents.

The thus obtained cross-linked polymer film can then be dried again.

After cross-linking, which results in the formation of a substantially mechanically stable polymer film (dry film or wet film), the film is cut or chopped into small particles, or by other suitable means, with the thickness of the film defining the thickness of the particles. According to an embodiment, the film can be first cut or chopped and the then milled to further reduce the size of the particles. For obtaining particles with a well-defined size distribution, the milled particles can be sieved.

The particles are longer and/or wider than their thickness. For example, the particles can have a length and a width (in terms of the maximum imaginary diameter in these dimensions as described above) which each are at least 2-times, particularly 3-times and typically at least 4-times as large as the thickness of the particles. In further embodiments, the particles can be flat shaped and elongated so that, for example, the particles can have a length which at least 3-times as large as the thickness of the particles, while the width of the particles is at least 2-times as large as the thickness of the particles. Particularly, the particles can have a length which at least 4-times as large as the thickness of the particles, while the width of the particles is at least 2-times as large as the thickness of the particles.

In a further process, the particles are dried to remove the solvent.

In a further process, a pharmaceutically active composition in form of a solution or dispersion is mixed with the dry particles. The pharmaceutically active compound included in the pharmaceutically active composition is taken up by the polymer matrix of the particles and bound to the polymer matrix by, for example, physicochemical interaction. Non-bounded pharmaceutically active compound can be finally washed out.

According to an embodiment, the volume or amount of the added pharmaceutically active composition is adjusted to the capability of the dry particles to adsorb the pharmaceutically active composition so that the pharmaceutically active composition is substantially completely absorbed by the particles. As a consequence, most of the pharmaceutically active compound, for example a protein, is completely included in the particles. To improve homogeneous uptake, the particles can be gently mixed with the pharmaceutically active composition that can be optionally added step-wise.

Non-bound pharmaceutically active compound can be removed by washing. This is, however, not needed.

The particles loaded with the pharmaceutically active compound can be dried to remove any volatile component such as liquid or solvent contained in the pharmaceutically active composition.

According to an embodiment, a drug-delivery composition includes an intermediate composition including flat-shaped particles of a covalently cross-linked polymer network in which proteins of a given type are dispersed and releasably bound. The polymer network forms a hydrophilic matrix of the particles. The particles have an aspect ratio of at least 2:1, particularly of at least 4:1, and an average particle size of from 100 nm to 50 μm. According to an embodiment, the polymer network comprises cross-linked glycosaminoglycans, particularly cross-linked hyaluronic acid.

### Storage

According to an embodiment, dry particles of the cross-linked polymer, which do not include a protein or another pharmaceutically active compound, can be stored. For example, the particles can be stored as dry powder in small amounts. It is possible to store the dry powder for a very long time, even for years.

According to an embodiment, the dry particles are mixed with the pharmaceutically active composition, which includes a solution, for example an aqueous solution, and a protein and/or another pharmaceutically active compound, directly prior to the intended application, for example by the patient or by the practitioner. In this case, a kit including the dry particles in form of a powder and the pharmaceutically active composition in form of a liquid is provided.

According to an embodiment, the dry particles mixed with the pharmaceutically active composition, which includes a solution, for example an aqueous solution, and a protein and/or another pharmaceutically active compound, is provided in a suitable manner which allows storage for a given time. Hence, a pre-mix including the particles wetted or moisturized with the pharmaceutically active composition is provided. Typically, the pre-mix is such that most of the solution of the pharmaceutically active composition is taken up by the particles so that the particles have substantially completely soaked or absorbed the pharmaceutically active composition. For preparing the pre-mix, the amount of the pharmaceutically active composition and the amount of the particles are selected accordingly. Such a pre-mix can be stored, for example, at low temperature, for example in a refrigerator.

According to an embodiment, which can be combined with any other embodiment described herein, the stored pre-mix or the mixture prepared immediately prior to use is dispersed in a liquid medium to prepare an applicable drug delivery solution or dispersion for injection or for topical application such as for topical ophthalmic applications. In further embodiments, the stored pre-mix or the mixture prepared immediately prior to use is mixed with a matrix material to form an ointment, cream, gel, lotion, or dispersion suitable for topical applications.

The present invention shall be described in more detail in the following Examples.

### EXAMPLE 1

A hyaluronic acid film of 50 gram (concentration: 2 mass %) was provided. This film was air-dried by elevated temperature (50° C., 2 hours) and afterwards was added 10 ml of 1:1 mixture of 3 M HCl and 50% glutaraldehyde solution. The reaction took place for 10 minutes according to the protocol. The obtained cross-linked hyaluronic acid system was extensively washed with pure water. Thereafter the resulting system was dried again at 50° C. to obtain a dry system.

The obtained macroscopic cross-linked hyaluronic acid plate was reduced to small pieces (size of pieces 2 to 9 millimeter as average diameter) by a scissor. The resulting pieces were milled to microparticle size by means of Pulverisette 23 (Fritsch GmbH, Germany) and sieved via a 50 micrometer pore sieve. Typical examples are given in FIG. 4.

To 75 mg cross-linked hyaluronic acid microparticles there are added 600 microliter of a Gamma-Globulin solution (25 mg/ml). The solution was completely taken up by the dry microparticles. Thereafter were taken two samples of 108 and 109 mg and the Gamma-Globulin release into phosphate buffered saline was measured. The result is shown in FIG. 3

### EXAMPLE 2

In Example 2, the following drug delivery composition according to the present invention (in the following referred to as “matrix formulation”) was prepared and used for the studies described in the following.

The matrix formulation is composed of hyaluronic acid microparticles which comprise IL-1ra as pharmaceutically active ingredient.

In Example 2; various concentrations of IL-1ra, i.e. 50 mg/ml or 2.5 mg/drop, 25 mg/ml or 1.25 mg/drop, and 5 mg/ml or 0.25 mg/drop (solutions A-C), were compared to a standard steroid control (fluorometholone) in rabbits undergoing photorefractive keratectomy (PRK) surgery. Each of solutions A-C had a total volume of 9.5 ml and each of solutions A-C contained 190 drops (190 drops×50 microliter=9.5 milliliter per vial). The final hyaluronic acid concentration (i.e. the hyaluronic acid concentration in the final matrix formulation) was 0.526% w/w, based on the total weight of the matrix formulation.

Each rabbit received PRK in both eyes at a 100 μm ablation depth. In each rabbit, the right eye (OD) served as the treated eye while the left eye (OS) served as control. As in the previous trials all rabbits received antibiotic eye drops simultaneously to the treatment to prevent the development of infections. In all cases the treatment occurred four times daily and was continued until epithelial closure was complete.

As before corneal clarity, forward scatter and haze measurement were used as out-come parameters. Haze formation and scattering was assessed via slit lamp exam by an ophthalmologist. The timing for re-epithelization to occur in both the treatment and control groups was measured.

In addition Pentacam technology was used to image the anterior segment of the eye via a rotating Scheimpflug process that supplied pictures in three dimensions, allowing for a precise measurement of the shape, thickness, and the contour of the cornea. Using this method, the center of the cornea, which is most critical for refractive surgery planning, is measured precisely because of this rotational imaging process. Lastly post-experiment histological studies of stroma, epithelium, and the interface of the two tissues were conducted.

FIG. 5 shows the results of corneal clarity and haze measurement in the various treatment groups. In FIG. 5, mean (SD) grades for subjective evaluation of haze formation at weeks 1 to 6 for the steroid, interleukin 1 receptor antagonist (IL-1a), and control groups. A standard clinical haze grading scale from 0 to 4 was used, with grade 0 representing a clear cornea, grade 1 representing trace haze, grade 2 representing mild haze, grade 3 representing moderate haze, and grade 4 representing severe haze. IL-1ra groups are combined because there was no statistically significant difference between them. Note the difference between the steroid group, the IL-1ra groups, and the control groups at weeks 4 and 5. Error bars indicate standard deviation.

The resolution of haze over time is the critical outcome parameter. The results in FIG. 5 show a dose dependent effect of haze resolution (higher inventive matrix formulation concentrations worse than lower concentrations) and a non-inferior effect on haze reduction in the matrix formulation treated rabbits when compared to the steroid controls.

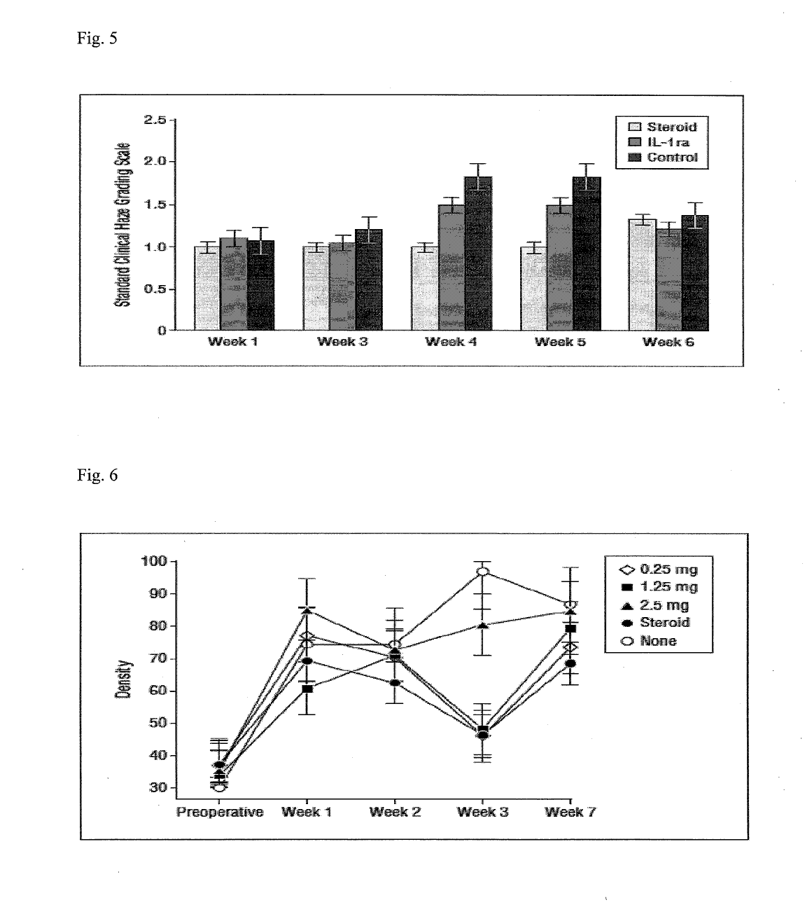

FIG. 6 summarizes the mean Central Optical Density (microns) among the various treatment groups when compared to steroids using Pentacam exams over time divided in left (OS) and right eye (OD). In FIG. 6, mean (SD) Pentacam density measurements between control (none) and treatment (2.5, 1.25, or 0.25 mg of interleukin 1 receptor antagonist (IL-1ra) groups before surgery (preoperative), at weeks 1 to 3 after surgery, and at week 7 after surgery. Note the statistically significant difference between the treatment groups with the lower doses of IL-1ra and the steroid group compared with the treatment group with the higher dose of IL_1ra and the control group at week 3. The trend continued through week 7 but was not statistically significant. Error bars indicate standard deviation.

A lower mean Central Optical Density is a desirable out-come. The results in FIG. 6 show again a dose dependent effect of the matrix formulation with higher doses resulting in lower mean peak densities when compared to steroids. In this study only the lowest dose of the matrix formulation was inferior to steroid control.

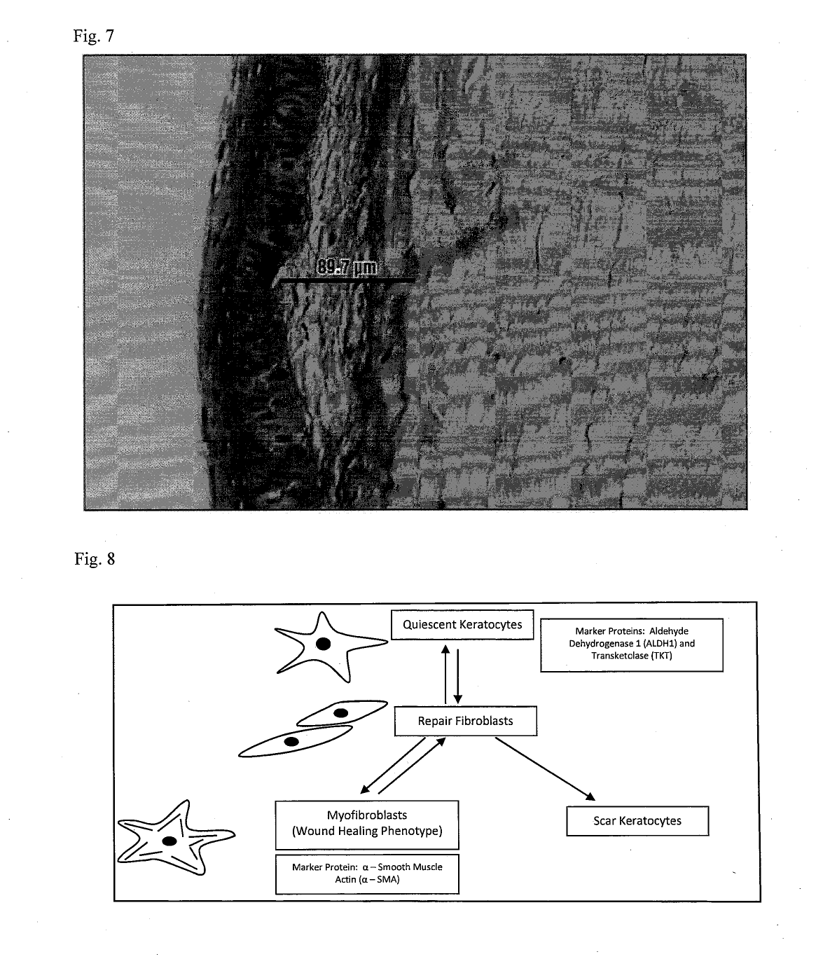

FIG. 7 summarizes the thickness of the foam layer (in microns) as an undesirable outcome among the various treatment groups when compared to steroids. FIG. 7 shows a hematoxylin-eosin stain of the cornea. The histologic subepithelial repair layer pictured was measured in every cornea by an ocular pathologist at both the central cornea and the thickest area of the foam layer. Thickness among the treatment groups was not statistically significant. The higher the number, the denser the foam layer so more light scattering and haze occurs. The matrix formulation was not inferior to steroids in all treatment groups.

In summary, the studies showed that the two lower concentrations of the matrix formulation proved to be as effective as steroids in controlling the inflammatory response. Surprisingly, the higher concentrations were actually less effective. The reason for this is not entirely clear, but the efficacy of the lower concentrations offers great benefits in both cost and the ability to package the drug in TheraKine's innovative timed-release technologies.

### EXAMPLE 3

In Example 3, a further rabbit study, similar to the design of Example 2 above, was performed using Novartis's canakinumab, a fully human antibody against Il-1 beta, incorporated in the matrix formulation using hyaluronic acid microparticles enhancing corneal adherence.

Even though it is described that canakinumab does differ from rabbit IL-1 in one amino acid, this study was performed to study the adherence characteristics, pk, and safety/tolerability of EpiKine delivering a different Il-1 inhibitor.

Preliminary results of this trial showed:
1) No adverse events with any of the concentrations of canakinumab. From a safety standpoint, canakinumab appeared to be non-inferior to steroids for the time frame it was administered in this study.
2) Although not statistically significant, there was a trend towards lower corneal haze at almost all data points measured with the 2.5 mg concentration of canakinumab.

### Epithelial Closure:

1) Compared to the steroid and control group, the canakinumab (all concentrations) took longer to heal, although not statistically significant.
2) Within the canakinumab concentrations, there was a trend for faster closure with the 1.25 mg concentration.
   Subjective Haze (slit lamp exam):
1) Canakinumab was as effective as steroids when compared to the control group.
2) At weeks 2, 4, 5, the canakinumab groups showed lower haze, although not statistically significant.
3) Within the canakinumab concentrations, the 2.5 mg concentration consistently showed a trend for lower subjective haze values.

### Objective Haze (Pentacam):

1) No difference in haze density at any time point for any of the groups.
2) Within the canakinumab concentrations, a trend for lower densitometry values in 2.5 mg concentration group was observed at each time point.
   Objective Haze (HRT stromal haze):
1) Although not statistically significant, the canakinumab group had less haze than the control group at every data point. Steroids had the least haze.
2) Within the canakinumab concentrations, the 2.5 mg showed statistically significant less haze as compared to control at one time point: week 2.

### Histologic Analysis:

1) There were no statistically significant differences in the formation of “foam layer” in any of groups.
2) In the canakinumab treated eyes, thinner foam layers were noted in eyes treated with the 2.5 mg and 1.25 mg concentration compared to 0.25 mg concentration.

### CONCLUSION

From this rabbit study, it was concluded that IL-1 inhibition also in the form of a human monoclonal IL-1 antibody (non inhibitory for rabbits) was safe and represented a valuable and promising alternative to topical steroids with a better side effect profile.

### EXAMPLE 4

### Matrix Formulation in Corneal Transplantation

The first step in studying the effects of the matrix formulation in corneal transplantation was to examine if it could control the redifferentiation of myofibroblasts back to the quiescent keratocyte phenotype.

### Effect of Blocking IL-1 on Cell Phenotype Over Time

Rabbit corneal fibroblasts were cultured over 21 days in tissue culture in media with and without the matrix formulation at a concentration of 30 μg/mL. Both freshly isolated and subcultured cells were tested in serum-containing medium and analyzed with Western Blot. The aim was to find a method to reduce smooth muscle actin (α-SMA) expression in subcultured corneal cells. Previous studies have suggested that abnormal corneal wound healing in patients after photorefractive keratectomy (PRK) is associated with the appearance of myofibroblasts in the stroma between two and four weeks after surgery. Myofibroblasts progenitor cells express SMA prior to completion of the differentiation pathway into corneal stromal cells. Therefore the purpose was to examine if the matrix formulation would suppress α-SMA expression and fibroblast differentiation.

It was found that matrix formulation had no effect on α-SMA expression in subcultured cells over the 21-day culture period. In addition, the matrix formulation significantly increased α-SMA expression in freshly isolated cells at days 2, 14 and 21 in culture.

### Effect of Blocking Interleukin-1 (IL-1) on Cell Phenotype in Media with and Without Serum

Since there is a variable amount of growth factors such as IL-1 in serum, the presence of serum may offset the effect of blocking IL-1 with the matrix formulation. In this experiment, freshly isolated and subcultured corneal fibroblasts (P2-P5) were seeded at a density of 5,000 cells/cm² and cultured with 30 μg/mL with human recombinant IL-1ra in either normal (NM) or Serum-Free (SF) media. Cells were analyzed on day 4 for levels of α-SMA with confocal microscopy. Cell numbers in serum-free samples were too low to recover enough protein for Western Blot analysis. 5 individuals independently counted 3 images of each of 3 samples per condition for α-SMA positive cells. Serum-free media induced lower α-SMA expression in freshly isolated cells but had no effect on subcultured cells. The combined effect of serum-free media and the matrix formulation significantly reduced α-SMA expression in both subcultured and freshly isolated cells on day 4 of culture. This finding supports that the phenotype of subcultured cells can be influenced by the matrix formulation.

Percentage of freshly isolated (FI) and subcultured (SC) RCFs expressing α-SMA as determined from confocal immunofluorescence on day 4 in the following conditions: normal media (NM), normal media with IL-1ra (NM+IL-1ra), serum-free (SF), and serum-free with IL-1ra (SF+IL-1ra). SF conditions decreased α-SMA levels in FI but not in SC cells. The application of IL-1ra in SF media significantly decreased levels of α-SMA in both FI and SC cells. Data represents mean+/−standard error with n=3 (* indicate p<<0.001, θ indicate p<0.05, φ indicate p<0.005).

### EXAMPLE 5

### Studies According to Example 4 Above were Further Expanded

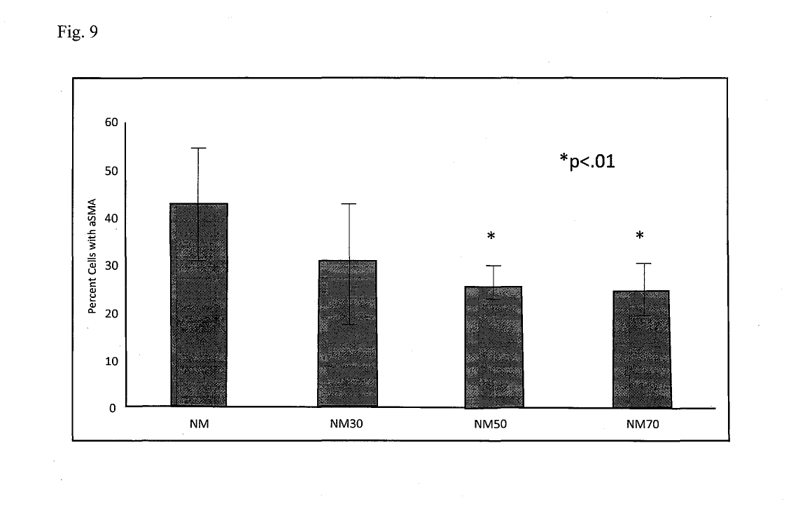

FIG. 9 illustrates preliminary confocal study results of cell grown in 3D collagen matrices with different amounts of the matrix formulation as prepared in Example 2. NM30=normal media plus 30 μg/mL IL-1ra. The matrix formulation indicates alpha-SMA protein reduction in 3D culture environment, which is essential for corneal tissue engineering.

### Summary of Results with EpiKine in an In vitro Corneal Model

Immunofluorescence analysis demonstrated that the combined effect of the matrix formulation in a serum-free media significantly reduced levels of α-SMA in both FI and SC cells. Such a treatment may lead to a reduced myofibroblast population and therefore reduced light scatter after VC surgery.

### SUMMARY

By Examples 1-5, the matrix formulation according to the present invention has shown beneficial effects in vitro and in vivo models of corneal injury and corneal transplantation.

These results indicate that IL-1 inhibition by topical use of the matrix formulation allows for a safe and effective treatment of corneal injuries. More specifically, the matrix formulation is not associated with an increased infection risk or delay in wound healing. IL-1 inhibition by topical use of the matrix formulation provides superior visual outcome through better corneal healing after LVC. The matrix formulation is non inferior and in many aspects superior to steroids, the current gold standard for the treatment of corneal healing. The matrix formulation improves safety outcomes by reducing infections & haze and offers the potential to improve long-term outcomes of wavefront LVC surgeries. The matrix formulation might be beneficial in corneal transplantation by significantly reducing levels of α-SMA in a corneal model.

### EXAMPLE 6

Release of antibodies from drug-delivery compositions according to the invention has been examined using different drug-delivery compositions according to embodiments described herein.

Particularly, hydrophilic matrices comprising cross-linked glycosaminoglycans, specifically cross-linked hyaluronic acid particles, have been used. In the following, the cross-linked hyaluronic acid particles are referred to as polymer particles.

The polymer particles are prepared as described in Example 1. 50 mg of the dried cross-linked polymer particles were given to 1 ml of a solution containing antibodies representing here a therapeutic protein. The table below lists the respective pharmaceutically active compositions (pharmaceutically active compound dispersed or solved in a liquid component) to which 50 mg of dry polymer matrix per 1 ml were given, so that the resulting intermediate compositions ED001 to ED006 were obtained. Each intermediate composition thus had 5 w/w % of polymer particles, and the weight ratio between the polymer particles and the pharmaceutically active compound (antibody) was 1:1. The polymer particles nearly completely absorbed the respective pharmaceutically active compositions so that swollen polymer particles were obtained which form the respective intermediate compositions

| Intermediate | |
|---|---|
| composition | Content |
| ED-001 | 5.0 w/w % protein, 0.2 w/w % Tween 80, 94.8 w/w % |
| | phosphate buffered saline (PBS, pH 7.4), 5.0 w/w % |
| | protein polymer particles |
| ED-002 | 5.0 w/w % protein, 0.1 w/w % Tween 80, 0.2 w/w % |
| | hyaluronic acid, 94.7 w/w % phosphate buffered saline |
| | (PBS, pH 7.4), 5.0 w/w % protein polymer particles |
| ED-003 | 5.0 w/w % protein, 0.1 w/w % trehalose, 94.9 w/w % |
| | phosphate buffered saline (PBS, pH 7.4), 5.0 w/w % |
| | protein polymer particles |
| ED-004 | 5.0 w/w % protein, 0.1 w/w % sodium dodecyl sulfate, |
| | 94.9 w/w % phosphate buffered saline (PBS, pH 7.4), |
| | 5.0 w/w % protein polymer particles |
| ED-005 | 5.0 w/w % protein, 0.1 w/w % Tween 80, 0.2 w/w % |
| | hyaluronic acid, 0.3 w/w % trehalose, 94.4 w/w % |
| | phosphate buffered saline (PBS, pH 7.4), 5.0 w/w % |
| | protein polymer particles |
| ED-006 | 5.0 w/w % protein, 95.0 w/w % phosphate buffered |
| | saline (PBS, pH 7.4), 5.0 w/w % protein polymer |
| | particles |

After washing steps in PBS at pH 7.4, the release of the antibodies from the swollen polymer particles were determined in PBS at pH 7.4 free of further components. The results are given in FIG. 10.

As can be gathered from FIG. 10, a sustained release of the antibodies can be observed. 50% of the antibodies are released after about 30 min depending on the composition of the intermediate composition.

ED-004 shows a comparably fast release with a complete release within 2 h. The other intermediate compositions show a constant release after 1 h at different rates. Hence, the release rate, after 1 h, is about between 50% per hour to about 5% per hour. It is therefore possible to adjust the release kinetics by adapting the liquid of the pharmaceutically active composition.

In the following, embodiments are described in itemized form:
1. Drug-delivery composition, comprising:
   a liquid medium; and
   an intermediate composition comprising flat shaped particles dispersed in the liquid medium, the particles comprise a hydrophilic matrix of covalently cross-linked glycosaminoglycans and a pharmaceutically active composition comprising a protein dispersed or distributed in the hydrophilic matrix of the particles, wherein the particles have an aspect ratio of 2:1 to 50:1 and an average particle size of 100 nm to 200 μm, particularly of 100 nm to 100 μm, and wherein the protein is an antibody or a therapeutic protein;
   wherein the drug-delivery composition is for one of topical applications, topical ophthalmic applications, and injections.
2. Drug-delivery composition for topical applications and injections, comprising:
   an intermediate composition, comprising
      a hydrophilic matrix, and
      a pharmaceutically active composition,
         said pharmaceutically active composition comprises a protein,
         said pharmaceutically active composition is dispersed or distributed in the hydrophilic matrix,
         said hydrophilic matrix is in form of particles,
         said particles comprise cross-linked hyaluronic acid,
         said particles have an aspect ratio of 1:1 to 50:1
         said particles have an average particle size of 100 nm to 200 μm, particularly of 100 nm to 100 μm.
3. Drug-delivery composition, comprising
   an intermediate eye drop composition, comprising
      a hydrophilic matrix, and
      a pharmaceutically active composition,
         said pharmaceutically active composition comprises a protein,
         said pharmaceutically active composition is dispersed or distributed in the hydrophilic matrix,
         said hydrophilic matrix is in form of particles,
            said particles comprise at least one cross-linked polymer,
            said particles have an aspect ratio of 1:1 to 50:1,
            said particles have an average particle size of 100 nm to 200 μm, particularly of 500 nm to 5 μm.
4. Drug-delivery composition, comprising
   an intermediate composition, comprising
      a hydrophilic matrix, and
      a pharmaceutically active composition,
         said pharmaceutically active composition comprises a protein, −said protein is an antibody or a therapeutic protein,
         said pharmaceutically active composition is dispersed or distributed in the hydrophilic matrix,
         said hydrophilic matrix is in form of particles,
            said particles comprise at least one cross-linked polymer,
            said particles have an aspect ratio of 1:1 to 50:1,
            said particles have an average particle size of 100 nm to 200 μm, particularly of 100 nm to 100 μm.
5. Drug-delivery composition, comprising
   an intermediate composition, comprising
      a hydrophilic matrix, and
      a pharmaceutically active composition,
         said pharmaceutically active composition comprises a protein or another pharmaceutically active compound,
         said pharmaceutically active composition is dispersed or distributed in the hydrophilic matrix,
         said hydrophilic matrix is in form of particles,
            said particles comprise at least one cross-linked polymer,
            said particles have an aspect ratio of 1:1 to 50:1,
            said particles have an average particle size of of 100 nm to 200 μm, particularly 100 nm to 100 μm.
6. Drug-delivery composition according to any one of claims 1-5, in form of an ointment, cream, gel, lotion, dispersion, solution or injection solution.

7. Drug-delivery composition according to any one of embodiments 1-6, wherein the concentration of intermediate composition or of the particles is from 0.1% w/w to 70% w/w, particularly from 1% w/w to 70% w/w based on the total weight of the drug-delivery composition.
8. Drug-delivery composition according to any one of embodiments 2-7, wherein the drug-delivery composition further comprises a liquid medium.
9. Drug-delivery composition according to embodiment 1 or 7, wherein the liquid medium is aqua ad injectabilia.
10. Drug-delivery composition according to any one of embodiments 1-9, wherein the drug-delivery composition further comprises at least one additive.
11. Drug-delivery composition according to embodiment 10, wherein the at least one additive is selected from the group consisting of a soothing agent, a buffer agent, a preservative agent, a surfactant, a stabilizing agent, a tonicity agent and an antioxidant.
12. Drug-delivery composition according to any one of embodiments 1-11, wherein the drug-delivery composition is a solution comprising the intermediate composition as solute, and the liquid medium as solvent.
13. Drug-delivery composition according to any one of embodiments 1-12, wherein the drug-delivery composition is a dispersion comprising the intermediate composition as dispersed phase, and the liquid medium as dispersant.
14. Drug-delivery composition according to embodiment 13, wherein the dispersed phase is a colloidal dispersed phase.
15. Drug-delivery composition according to any one of embodiments 1-14, wherein the drug-delivery composition has a pH value of 6.5 to 7.5, particularly of 7.3 to 7.5, and an osmolarity of 250 mosmol/l to 350 mosmol/l.
16. Drug-delivery composition according to any one of embodiments 1-15, wherein the drug-delivery composition is in form of liquid eye drops, oily eye drops, eye baths, eye gels, eye ointments, eye creams.
17. Drug-delivery composition according to any one of embodiments 1-16 for use in the treatment of eye disorders.
18. Drug-delivery composition according to embodiment 17, wherein eye disorders are selected from inflammatory eye disorders, traumatic injuries of the cornea, such as shrapnel injuries, non-infectious corneal ulcers, surgical procedures like laser vision correction, or corneal transplants.
19. Drug-delivery composition according to any one of embodiments 1-18, wherein the drug-delivery composition shows sustained release of the protein.
20. Drug-delivery composition according to any one of embodiments 1-19, wherein the weight ratio between hydrophilic matrix and the protein is from 4:1 to 200:1, or from 1:1 to 10:1.
21. Drug-delivery composition according to any one of embodiments 1-20, wherein the cross-linked polymer is a naturally occurring polymer.
22. Drug-delivery composition according, to any one of embodiments 1-21, wherein the cross-linked polymer is selected from hyaluronic acid, fibrin, polyvinyl alcohol (PVA).
23. Drug-delivery composition according to any one of embodiments 1-22, wherein the pharmaceutically active composition comprises a liquid component selected from the group consisting of hydrophilic solvents, lipophilic solvents and solubilizers, or in any combination of two or more kinds thereof.
24. Drug-delivery composition according to any one of embodiments 1-23, wherein the pharmaceutically active composition is a dispersion comprising the protein as colloidal dispersed phase, and the liquid component as dispersant.
25. Drug-delivery composition according to any one of embodiments 1-24, wherein the protein comprised in the pharmaceutically active composition is a naturally occurring protein and/or a competitive receptor antagonist of IL-1 or a competitive antagonist of IL-1 alpha and or beta.
26. Drug-delivery composition according to any one of embodiments 1-25, wherein the protein is IL-1ra or IL-1 Mab, IL-1 trap or anti IL-1 fusion protein.
27. Drug-delivery composition according to any one of embodiments 2-25, wherein the particles have an aspect ratio of 2:1 to 50:1.
28. Method for manufacturing a drug-delivery composition, comprising
   providing a hydrophilic matrix, comprising at least one cross-linked polymer, in form of particles;
   providing a pharmaceutically active composition comprising a protein; and
   mixing the hydrophilic matrix and the pharmaceutically active composition so that the protein is provided in a weight ratio from 1:4 to 1:200, or from 1:1 to 1:10 relative to the hydrophilic matrix to form an intermediate composition of the drug-delivery composition, wherein the intermediate composition comprises the particles and the protein distributed within the particles.
29. Method according to embodiment 28, wherein providing a hydrophilic matrix comprises providing a film of non-cross-linked polymer having a molecular weight of at least 10000 Da, and cross-linking the polymer of said film.
30. Method according to embodiment 28 or 29, wherein the concentration of non-cross-linked polymer in the film, particularly the wet film, is from 0.1 mass % to 20 mass %, particularly from 0.1 mass % to 10 mass % based on the total mass of the film, the method further comprises:
   drying the film of the non-cross-linked polymer at an elevated temperature of from 30° C. to 70° C., for 0.5 hour to 10 hours in order to obtain a dried film before cross-linking.
31. Method according to embodiment 30, wherein providing a hydrophilic matrix further comprises, after cross-linking, a further drying step performed at an elevated temperature of from 30° C. to 70° C. for 0.5 hour to 5 hours, particularly for 1 hour to 3 hours, typically for 2 hours to obtain a dried cross-linked material.
32. Method according to any one of embodiments 29-31, wherein providing a hydrophilic matrix further comprises breaking the cross-linked polymer material to particles by a mechanical process.
33. Method according to embodiment 32, wherein breaking the cross-linked polymer comprises a milling step carried out in a ball mill.
34. Method according to embodiment 32 or 33, further comprises sieving the particles using a metal sieve having a pore size of from 30 μm to 150 μm, particularly from 30 μm to 60 μm.
35. Method according to any one of embodiments 29-34, wherein the non-crosslinked polymer is a glycosaminoglycan.
36. Method according to any one of embodiments 28-35, wherein the protein is an antibody.
37. Method for manufacturing a drug-delivery composition, comprising:
   providing an liquid film of a non-cross-linked polymer;
   drying the liquid film of the non-cross-linked polymer to obtain an at least partially dried film of the non-cross-linked polymer having a moisture content of less than 30%;
   adding a cross-linking agent to the at least partially dried film of the non-cross-linked polymer to cross-link the polymer;
   drying the film of the cross-linked polymer at an elevated temperature to obtain a dried film of the non-cross-linked polymer; and
   breaking the dried film of the cross-linked polymer to particles by a mechanical process to obtain particles having an aspect ratio of 1:1 to 50:1, particularly of 2:1 to 50:1, and an average particle size of 100 nm to 200 μm, particularly of 100 nm to 100 μm.
38. Method according to embodiment 37, wherein the concentration of non-cross-linked polymer in the aqueous film is from 0.1 mass % to 20 mass %, particularly from 0.1 mass % to 10 mass % based on the total mass of the aqueous film.
39. Method according to embodiment 37 or 38, wherein the non-cross-linked polymer has a molecular weight of at least 10000 Da.
40. Method according to any of the embodiments 27-39, wherein drying the aqueous film of the non-cross-linked polymer is carried out at a temperature of from 30° C. to 70° C., for 0.5 hour to 10 hours.
41. Method according to any of the embodiments 37-40, wherein drying the film of the cross-linked polymer is carried out at a temperature from 30° C. to 70° C. for 0.5 hour to 5 hours, particularly for 1 hour to 3 hours, typically for 2 hours.
42. Method according to any of the embodiments 37-41, wherein the polymer is a polysaccharide.
43. Method according to any of the embodiments 37-42, wherein the polymer is a glycosaminoglycans.
44. Method according to any of the embodiments 37-43, further comprising:
   providing a pharmaceutically active composition comprising a solution and a protein,
   mixing the particles and the pharmaceutically active composition so that the protein is adsorbed in the particles in a weight ratio from 1:4 to 1:200, or a weight ratio from 1:1 to 1:10 relative to the dry hydrophilic matrix of the particles to form an intermediate composition of the drug-delivery composition.
45. Method according to embodiment 44, wherein the pharmaceutically active composition and the particles are provided in respective amounts relative to each other so that that pharmaceutically active composition is substantially completely adsorbed by the particles.
46. Drug-delivery composition which is obtainable by a method according to any of the embodiments 28-45.
47. Treatment of an eye disease by administering a drug-delivery composition of any of the embodiments 1-27.

1-42. (canceled)

43. A drug-delivery composition, comprising:
an intermediate composition, comprising
a hydrophilic matrix, wherein the hydrophilic matrix is in form of particles, said particles comprise at least one cross-linked polymer, said particles having an aspect ratio of about 1:1 to about 50:1 or about 2:1 to about 50:1, said particles having an average particle size of about 100 nm to about 200 μm, preferably having an average particle size of about 100 nm to about 100 μm; and
a pharmaceutically active composition, wherein said pharmaceutically active composition comprises a protein or another pharmaceutically active compound, wherein said pharmaceutically active composition is dispersed or distributed in the hydrophilic matrix.

44. The drug-delivery composition of claim 43, further comprising:
a liquid medium, said liquid medium comprising the intermediate composition.

45. The drug-delivery composition of claim 44, wherein the intermediate composition comprises flat shaped particles dispersed in the liquid medium.

46. The drug-delivery composition of claim 44, wherein the liquid medium is aqua ad injectabilia.

47. The drug-delivery composition of claim 44, wherein the drug-delivery composition is a solution, said solution comprising the intermediate composition as solute and the liquid medium as a solvent.

48. The drug-delivery composition of claim 44, wherein the drug-delivery composition is a dispersion, said dispersion comprising the intermediate composition as dispersed phase and the liquid medium as a dispersant.

49. The drug-delivery composition of claim 48, wherein the dispersed phase is a colloidal dispersed phase.

50. The drug-delivery composition of claim 43, further comprising:
at least one additive.

51. The drug-delivery composition of claim 50, wherein the additive is selected from the group consisting of a soothing agent, a buffer agent, a preservative agent, a surfactant, a stabilizing agent, a tonicity agent and an antioxidant.

52. The drug-delivery composition of claim 43, wherein the hydrophilic matrix comprises covalently cross-linked glycosaminoglycans.

53. The drug-delivery composition of claim 43, wherein the protein is a naturally occurring protein or a synthetically derived protein, wherein the protein is an antibody, a therapeutic protein, a competitive receptor antagonist of IL-1, a competitive antagonist of IL-1 alpha, a competitive antagonist of IL-1 beta, IL-1 receptor antagonist, IL-1 monoclonal antibody, anti-IL-1 fusion protein or a combination thereof.

54. The drug-delivery composition of claim 43, wherein said drug-delivery composition is administered by topical route, ophthalmic topical route or as an injection.

55. The drug-delivery composition of claim 43, wherein formulation of said drug-delivery composition is selected from the group consisting of an ointment, a cream, a gel, a lotion, a dispersion, a solution and an injection.

56. The drug-delivery composition of claim 43, wherein concentration of the intermediate composition or of the particles is about 0.1% w/w to about 70% w/w, preferably from about 1% w/w to about 70% w/w based on the total weight of the drug-delivery composition.

57. The drug-delivery composition of claim 43, wherein the drug-delivery composition has a pH value of about 6.5 to about 7.5, preferably about 7.3 to about 7.5, and an osmolarity of about 250 mosmol/l to about 350 mosmol/l.

58. The drug-delivery composition of claim 43, wherein the drug-delivery composition is in the form selected from the group consisting of liquid eye drops, oily eye drops, eye baths, eye gels, eye ointments and eye creams.

59. The drug-delivery composition of claim 43, wherein said drug-delivery composition is used in the treatment of an eye disorder, wherein said eye disorder is selected from the group consisting of an inflammatory eye disorder, a traumatic injury of the cornea, a shrapnel injury, a non-infectious corneal ulcer, and a surgical procedure, wherein said surgical procedure is a laser vision correction or a corneal transplant.

60. The drug-delivery composition of claim 43, wherein the drug-delivery composition allows sustained release of the protein.

61. The drug-delivery composition of claim 43, wherein the weight ratio between the hydrophilic matrix and the protein is from about 1:1 to about 10:1 or from about 4:1 to about 200:1.

62. The drug-delivery composition of claim 43, wherein the cross-linked polymer is a naturally occurring polymer or a synthetically-derived polymer, said cross-linked polymer is selected from the group consisting of hyaluronic acid, fibrin, and polyvinyl alcohol (PVA).

63. The drug-delivery composition of claim 43, wherein the pharmaceutically active composition comprises a liquid component, said liquid component selected from the group consisting of hydrophilic solvents, lipophilic solvents and solubilizers or a combination thereof.

64. A method of treating an eye disease, comprising:
administering the drug-delivery composition of claim 43.

65. A method for manufacturing a drug-delivery composition, comprising:
providing a hydrophilic matrix by providing a film of non-cross-linked polymer having a molecular weight of at least 10,000 Da, and cross-linking the polymer of the film such that said hydrophilic matrix comprises at least one cross-linked polymer in form of particles, wherein the non-cross-linked polymer is a glycosaminoglycan;
providing a pharmaceutically active composition comprising a protein; and
mixing the hydrophilic matrix and the pharmaceutically active composition so that the protein is provided in a weight ratio from about 1:1 to about 1:10, or from about 1:4 to about 1:200, relative to the hydrophilic matrix to form an intermediate composition of the drug-delivery composition, wherein the intermediate composition comprises the particles and the protein distributed within the particles.

66. The method of claim 65, wherein the concentration of non-cross-linked polymer in the film is from about 0.1 mass % to about 20 mass %, preferably from about 0.1 mass % to about 10 mass % based on the total mass of the film, said method further comprising:
drying the film of the non-cross-linked polymer at an elevated temperature of from about 30° C. to about 70° C., for about 0.5 hour to about 10 hours to obtain a dried film before cross-linking.

67. The method of claim 66, wherein providing the hydrophilic matrix further comprises:
performing a drying step after the cross-linking, said drying step is performed at an elevated temperature of from about 30° C. to about 70° C., for about 0.5 hour to about 5 hours, preferably for about 1 hour to 3 hours, preferably for about 2 hours to obtain a dried cross-linked material.

68. The method of claim 65, wherein providing the hydrophilic matrix further comprises:
breaking the cross-linked polymer to particles by a mechanical process.

69. The method of claim 68, wherein breaking the cross-linked polymer comprises a milling step carried out in a ball mill.

70. The method of claim 68, further comprising:
sieving the particles using a metal sieve having a pore size of from about 30 μm to about 150 μm, preferably from about 30 μm to about 60 μm.

71. The method of claim 65, wherein the protein is an antibody.

72. A method for manufacturing a drug-delivery composition, comprising:
providing a liquid film of a non-cross-linked polymer;
drying the liquid film of the non-cross-linked polymer to obtain at least
partially dried film of the non-cross-linked polymer having a moisture content of less than about 30%;
adding a cross-linking agent to the partially dried film of the non-cross-linked polymer to cross-link the polymer;
drying the film of the cross-linked polymer at an elevated temperature to obtain a dried film of the non-cross-linked polymer; and
breaking the dried film of the cross-linked polymer to particles by a mechanical process to obtain particles having an aspect ratio of about 1:1 to about 50:1, preferably about 2:1 to about 50:1, and an average particle size of about 100 nm to about 200 μm, preferably about 100 nm to about 100 μm.

73. The method of claim 72, further comprising:
providing a pharmaceutically active composition comprising a solution and a protein; and
mixing the particles and the pharmaceutically active composition so that the protein is adsorbed in the particles in a weight ratio from about 1:1 to about 1:10, or a weight ratio from about 1:4 to about 1:200, relative to the dry hydrophilic matrix of the particles to form an intermediate composition of the drug-delivery composition.

74. The method of claim 73, wherein the pharmaceutically active composition and the particles are provided in respective amounts relative to each other so that the pharmaceutically active composition is substantially completely adsorbed by the particles.

75. The method of claim 72, wherein the concentration of the non-cross-linked polymer in the aqueous film is from about 0.1 mass % to about 20 mass %, preferably from about 0.1 mass % to about 10 mass % based on the total mass of the aqueous film.

76. The method of claim 72, wherein the non-cross-linked polymer has a molecular weight of at least 10,000 Da.

77. The method of claim 72, wherein drying the aqueous film of the non-cross-linked polymer is carried out at a temperature from about 30° C. to about 70° C., for about 0.5 hour to about 10 hours.

78. The method of claim 72, wherein drying the aqueous film of the non-cross-linked polymer is carried out at a temperature from about 30° C. to about 70° C., for about 0.5 hour to about 5 hours, preferably for about 2 hours.

79. The method of claim 72, wherein the polymer is a polysaccharide or a glycosaminoglycan.

80. A drug-delivery composition obtained by the method of claim 72.
